(19) United States
(12) Patent Application Publication
(10) Pub. No.: US 2026/0298889 A1
(43) Pub. Date: Oct. 1, 2026

(54) SIGNAL AMPLIFICATION FOR MASS DETECTION

(71) Applicant: Donald Danforth Plant Science Center, St. Louis, MO (US)

(72) Inventors: Allen Hubbard, St. Louis, MO (US); Ivan Baxter, St. Louis, MO (US); Louis Connelly, St. Louis, MO (US)

(21) Appl. No.: 19/489,362

(22) PCT Filed: Jun. 3, 2024
(86) PCT No.: PCT/US2024/032259

§ 371 Date: Dec. 2, 2025

Related U.S. Application Data
(60) Provisional application No. 63/505,941, filed on Jun. 2, 2023.

(51) Int. Cl. G01N 30/72; G01N 30/02; G01N 30/86; G01N 30/88; G16C 20/20

(52) U.S. Cl. CPC G01N 30/7233; G01N 30/8658; G01N 30/88; G16C 20/20; G01N 2030/027; G01N 2030/8813

(57) ABSTRACT

Disclosed embodiments provide a framework for the amplification of signals to detect metabolites from a set of samples without isotopologues. A plurality of samples is combined into a merged mass spectrum of mass-to-charge (m/z) signal intensities. From the merged mass spectrum, a first set of metabolites are identified based on a concentration of signals corresponding to the first set of metabolites and corresponding isotopologues. A signal bandwidth is calculated based on a limit of detection for the merged mass spectrum and a lowest signal intensity corresponding to an isotopologue associated with the first set of metabolites. From the signal bandwidth, a set of signal intensity adjacencies are identified that are indicative of a second set of metabolites within the merged mass spectrum.

### CROSS-REFERENCE TO RELATED APPLICATIONS

This application claims priority from Provisional Application No. 63/505,941, filed Jun. 2, 2023, the entire contents of which are hereby incorporated by reference.

### GOVERNMENTAL RIGHTS

This invention was made with government support under DE-SC0018277 and DESC0023160 awarded by the Department of Energy. The government has certain rights in the invention.

### FIELD OF THE INVENTION

The present disclosure relates generally to compound detection from chromatography-mass spectrometry (chromatography-MS) samples. More specifically, the present disclosure relates to the amplification of signals to detect compounds from a set of samples without isotopologues.

### BACKGROUND OF THE INVENTION

LC-MS is a chemical technique that relies on two dimensions of separation to identify different compounds in a sample as unique mass features. A liquid chromatography system may separate the different compounds by structural properties, while a mass spectrometer subsequently determines the mass and intensity (e.g., mass-to-charge or m/z signals) of the ions that elute from the chromatography column. Modern high-resolution mass spectrometry can now detect and quantify ions with high mass precision (<5 ppm mass error) but may also result in significant amounts of noise. Thus, one of the greatest challenges in harnessing LC-MS for untargeted metabolomics, especially at the level of reliability required for clinical applications, is being able to overcome signal-to-noise challenges. For instance, researchers are often concerned with detecting the signatures of faint but biologically important metabolites without spuriously misclassifying noise signals as metabolites.

Some current solutions for validating signals of metabolite masses via the paired signatures of M0 and M1 peaks have been implemented through scalable algorithms in the context of a merged mass spectrum. This merged mass spectrum amplifies the signal-to-noise such that normally undetectable M1 peaks emerge, so long as these peaks are above the limit of detection in at least some of the samples. These current solutions have proven to be fairly robust. However, these current solutions fail to address a particular gap that becomes more pronounced in the emerging context of clinical metabolomics (and other situations where analyte concentrations are faint (i.e. single cell metabolomics)). This particular gap corresponds to important metabolites which are detectable in human serum and that are present at levels low enough that the M1 peaks are below detection in all samples. As a result, these current solutions fail to detect these masses.

Thus, there is a need for improved systems and methods of identifying metabolite signals accurately from datasets of hundreds to thousands of samples in any species without misclassifying signals corresponding to noise or inorganic salts as being metabolites, even when M1 peaks are unavailable.

### SUMMARY OF THE INVENTION

One aspect of the instant disclosure encompasses a method for detecting compounds with weak signals in mass spectrometry (MS) data. The method comprises (a) combining or having combined a plurality of chromatography-mass spectrometry (chromatography-MS) mass spectra comprising mass-to-charge (m/z) signal intensities associated with mass measurements of compounds into a merged mass spectrum of m/z signal intensities; (b) identifying a first set of compounds from the merged mass spectrum, wherein the first set of compounds is identified based on a concentration of signals corresponding to mass measurements associated with the first set of compounds and isotopologues of the first set of compounds; (c) calculating a signal bandwidth for identification of a second set of compounds, wherein the signal bandwidth is calculated based on a limit of detection associated with the mass spectrometer and a lowest signal intensity corresponding to an isotopologue associated with the first set of compounds; and (d) identifying a set of signal intensity adjacencies within the signal bandwidth, wherein the set of signal intensity adjacencies are indicative of a second set of compounds. The method can optionally comprise filtering signals associated with the first set of compounds from the merged mass spectrum prior to identifying the set of signal intensity adjacencies within the signal bandwidth. The method can also optionally comprise iteratively repeating steps (b) to (d). Importantly, the second set of compounds can be identified without requiring identification of corresponding isotopologues from the merged mass spectrum.

In some embodiments, the method further comprises receiving or having received the plurality of chromatography-MS mass spectra. In some embodiments, the compound is metabolites, isomers of metabolites, or both. Additionally, the chromatography-MS can be liquid chromatography-MS (LC-MS).

In some embodiments, a method of the instant disclosure further comprises dividing the merged mass spectrum within the signal bandwidth into bins, wherein the bins are sized to detect the set of signal intensity adjacencies within the signal bandwidth. In some embodiments, the set of signal intensity adjacencies are identified according to statistical differences between known metabolite signal intensities and electrostatic noise signal intensities. In some embodiments, the bins correspond to an atomic mass range equivalent to 1/10,000th Daltons.

The method can also further comprise identifying one or more inorganic salts from the merged mass spectrum, wherein the one or more inorganic salts are identified as a result of signal intensities corresponding to the one or more inorganic salts exceeding a required lowest signal intensity corresponding to an expected isotopologue, while there is no expected isotopologue detected.

In some embodiments, the set of signal intensity adjacencies are identified according to statistical differences between known metabolite signal intensities and electrostatic noise signal intensities. Further, the second set of compounds can be identified without requiring identification of corresponding isotopologues from the merged mass spectrum.

Another aspect of the instant disclosure encompasses a computing system for detecting compounds with weak signals in mass spectrometry (MS) data. The computing system comprises: (a) a communication interface that receives EICs of portions of each of a plurality of MS chromatograms, wherein the portions of the plurality of chromatograms bear the same m/z value; and (b) a processor that executes instructions stored in memory, wherein the processor executes the instructions to: (i) identify a first set of compounds from a merged mass spectrum of m/z signal intensities, wherein the first set of compounds is identified based on a concentration of signals corresponding to mass measurements associated with the first set of compounds and isotopologues of the first set of compounds; (ii) calculate a signal bandwidth for identification of a second set of compounds, wherein the signal bandwidth is calculated based on a limit of detection associated with the mass spectrometer and a lowest signal intensity corresponding to an isotopologue associated with the first set of compounds; and (iii) identify a set of signal intensity adjacencies within the signal bandwidth, wherein the set of signal intensity adjacencies are indicative of a second set of compounds; (iv) optionally filter signals associated with the first set of compounds from the merged mass spectrum prior to identifying the set of signal intensity adjacencies within the signal bandwidth; and (v) optionally iteratively repeat steps (i) to (iv). In some embodiments, the compound is metabolites, isomers of metabolites, or both. In some embodiments, the chromatography-MS is liquid chromatography-MS (LC-MS). The ceiling for this ideal signal bandwidth can be dynamically calculated by identifying the signal intensity corresponding to the least abundant metabolite that is part of a valid isopair. In some embodiments, the processor executes the instructions to receive the plurality of chromatography-MS mass spectra.

In some embodiments, the processor further executes the instructions to: divide the merged mass spectrum within the signal bandwidth into bins. The bins can be sized to detect the set of signal intensity adjacencies within the signal bandwidth. In some embodiments, the bins correspond to an atomic mass range equivalent to 1/10,000th Daltons.

The processor can further execute the instructions to: identify one or more inorganic salts from the merged mass spectrum. The one or more inorganic salts can be identified as a result of signal intensities corresponding to the one or more inorganic salts exceeding a required lowest signal intensity corresponding to an expected isotopologue, while there is no expected isotopologue detected.

In some embodiments, the set of signal intensity adjacencies are identified according to statistical differences between known metabolite signal intensities and electrostatic noise signal intensities.

In some embodiments, the second set of compounds is identified without requiring identification of corresponding isotopologues from the merged mass spectrum.

Yet another aspect of the instant disclosure encompasses a non-transitory, computer-readable storage medium, having embodied thereon a program executable by a processor to perform a method of detecting compounds with weak signals in mass spectrometry (MS) data. The method comprises (a) combining or having combined a plurality of chromatography-mass spectrometry (chromatography-MS) mass spectra comprising mass-to-charge (m/z) signal intensities associated with mass measurements of compounds into a merged mass spectrum of m/z signal intensities; (b) identifying a first set of compounds from the merged mass spectrum, wherein the first set of compounds is identified based on a concentration of signals corresponding to mass measurements associated with the first set of compounds and isotopologues of the first set of compounds; (c) calculating a signal bandwidth for identification of a second set of compounds, wherein the signal bandwidth is calculated based on a limit of detection associated with the mass spectrometer and a lowest signal intensity corresponding to an isotopologue associated with the first set of compounds; and (d) identifying a set of signal intensity adjacencies within the signal bandwidth, wherein the set of signal intensity adjacencies are indicative of a second set of compounds. The method can optionally comprise filtering signals associated with the first set of compounds from the merged mass spectrum prior to identifying the set of signal intensity adjacencies within the signal bandwidth. The method can also optionally comprise iteratively repeating steps (b) to (d). Importantly, the second set of compounds can be identified without requiring identification of corresponding isotopologues from the merged mass spectrum.

In some embodiments, the method further comprises receiving or having received the plurality of chromatography-MS mass spectra. In some embodiments, the compound is metabolites, isomers of metabolites, or both. Additionally, the chromatography-MS can be liquid chromatography-MS (LC-MS).

In some embodiments, a method of the instant disclosure further comprises dividing the merged mass spectrum within the signal bandwidth into bins, wherein the bins are sized to detect the set of signal intensity adjacencies within the signal bandwidth. In some embodiments, the set of signal intensity adjacencies are identified according to statistical differences between known metabolite signal intensities and electrostatic noise signal intensities. In some embodiments, the bins correspond to an atomic mass range equivalent to 1/10,000th Daltons.

The method can also further comprise identifying one or more inorganic salts from the merged mass spectrum, wherein the one or more inorganic salts are identified as a result of signal intensities corresponding to the one or more inorganic salts exceeding a required lowest signal intensity corresponding to an expected isotopologue, while there is no expected isotopologue detected.

In some embodiments, the set of signal intensity adjacencies are identified according to statistical differences between known metabolite signal intensities and electrostatic noise signal intensities. Further, the second set of compounds can be identified without requiring identification of corresponding isotopologues from the merged mass spectrum.

### BRIEF DESCRIPTION OF THE FIGURES

The following drawings form part of the present specification and are included to further demonstrate certain embodiments of the present disclosure. Certain embodiments can be better understood by reference to one or more of these drawings in combination with the detailed description of specific embodiments presented herein. The patent or application file contains at least one drawing executed in color. Copies of this patent or patent application publication with color drawing(s) will be provided by the Office upon request and payment of the necessary fee.

FIG. 1 shows an illustrative example of an environment in which a signal bandwidth within a merged mass spectrum is defined and from which valid metabolite masses can be identified without M1 isotopologues in accordance with at least one embodiment.

FIG. 2 shows an illustrative example of an environment in which metabolites with M0-M1 pairs and inorganic salts are filtered out to allow for identification of metabolite masses without M1 isotopologues in accordance with at least one embodiment.

FIG. 3 shows an illustrative example of an environment in which signal distributions within a defined signal bandwidth are evaluated to identify metabolite masses without M1 isotopologues in accordance with at least one embodiment.

FIG. 4 shows an illustrative example of an environment in which adjacently filled bins of the mass spectrum are identified through mapping signals to adjacent regions with a particular mass shift (corresponding to the bin width) in accordance with at least one embodiment.

FIG. 5 shows an illustrative example of a process for evaluating bins within a merged mass spectrum to identify metabolite masses without M1 isotopologues in accordance with at least one embodiment.

FIG. 6 shows an illustrative example of an environment in which various embodiments can be implemented.

### DETAILED DESCRIPTION

Embodiments of the present disclosure include systems and methods for detecting compounds with weak signals in mass spectrometry (MS) data. The present disclosure introduces an approach that overcomes the aforementioned limitations of relying on M1 peaks when such peaks fall below the threshold of detection in all samples. Methods of the instant disclosure utilize statistical characteristics of a merged mass spectrum to detect low-abundance compounds without the need for isotopologues. This method defines a signal bandwidth within the merged mass spectrum, which is selected to exclude the majority of noise signals and inorganic salts, thereby reducing the likelihood of false positives. The disclosed technique is highly scalable and has been successfully implemented on large datasets, demonstrating efficacy in identifying metabolites that were previously undetectable using conventional methods. This method can be especially useful in clinical metabolomics and other fields where precise metabolite detection is essential.

The ensuing description provides preferred examples of embodiment(s) only and is not intended to limit the scope, applicability or configuration of the disclosure. Rather, the ensuing description of the preferred examples of embodiment(s) will provide those skilled in the art with an enabling description for implementing preferred examples of embodiment. It is understood that various changes can be made in the function and arrangement of elements without departing from the spirit and scope as set forth in the appended claims.

### I. Method

One aspect of the present disclosure encompasses a method for detecting compounds with weak signals in mass spectrometry (MS) data. The method comprises calculating a signal bandwidth for identification of a second set of compounds by first identifying a first set of compounds from a merged mass spectrum of m/z signal intensities based on a limit of detection associated with the mass spectrometer and a lowest signal intensity corresponding to an isotopologue associated with the first set of compounds. The methods then identifies a second set of compounds by identifying a set of signal intensity adjacencies within the signal bandwidth, wherein the set of signal intensity adjacencies are indicative of a second set of compounds.

### (a) Mass Spectrometry

MS is a powerful analytical tool that can be combined with separation methods such as chromatography (chromatography-MS) for effective detection and quantitation of various compounds in a sample. Chromatography-MS comprises a first separation of components of a sample by chromatography where compounds with different properties separate and are collected at different times. The time point at which a certain fraction of a compound elutes from the column is called the retention time (RT). The resulting fractions are then inserted online into the mass spectrometer. Here the second separation takes place. By ionizing the compounds and accelerating them to fly through a spectrometer, the compounds are separated by their mass-to-charge (m/z) ratio (also referred to herein as “mass” for the sake of simplicity). Accordingly, a plurality of m/z signal intensities may be captured by a mass spectrometer in an output file, while intensity data records the abundance of a species of a given m/z relative to retention time. The measured data can then be depicted in a total ion chromatogram (TIC) 3D image comprising three axes. One axis depicts the RT, the second represents the m/z value, and the third axis represents the intensity or quantity of a peptide. The combined data of RT, m/z value, and intensity for each sample can be obtained as a chromatography-MS data file.

In some embodiments, the methods comprise obtaining or having obtained one or more data files from a chromatography-mass spectrometry machine that has analyzed samples of unknown compounds. The data in the data files can include a retention time and a mass-to-charge (m/z) signal intensity for each data point in the data file. In some embodiments, a method of the instant disclosure comprises combining the data files into a merged mass spectrum data file. The merged mass spectrum can amplify signals within the various samples. This is achieved by leveraging the statistical properties of the merged mass spectrum. When multiple samples are combined into a single merged mass spectrum, the true signals corresponding to metabolites or other compounds tend to hyper-concentrate along Gaussian or other non-random distributions. This hyper-concentration occurs because the true signals are consistent across multiple samples, whereas noise signals are more random and less likely to align consistently. In practical terms, this means that the merged mass spectrum inherently amplifies the true signals relative to the noise. For example, if a metabolite is present in multiple samples, its signal will appear at the same mass-to-charge (m/z) ratio in each sample. When these samples are combined, the signal for this metabolite will be reinforced, making it more prominent in the merged mass spectrum. In contrast, noise signals, which are random and inconsistent, will not reinforce each other in the same way and will remain relatively diffuse. This approach eliminates the need for traditional signal filtering techniques, which often involve setting arbitrary thresholds that can inadvertently exclude faint but biologically important signals. Instead, by focusing on the statistical properties of the merged mass spectrum, the method can enhance the detection of low-abundance metabolites without the risk of losing important data. This is particularly valuable in clinical metabolomics and other applications where the accurate detection of low-abundance compounds is critical.

The plurality of chromatograms can originate from a diverse range of samples, offering a broad scope for analysis and comparison. These chromatograms can be derived from a single sample, providing a detailed, focused examination of its components. Alternatively, chromatograms can be obtained from multiple samples collected independently, allowing for a comparative analysis across a wider range of variables. This versatility is particularly useful in experiments involving various treatments or conditions. For instance, samples from different treatment groups in a study can be analyzed to compare and contrast the effects of these treatments at a molecular level. Similarly, samples under different experimental conditions can provide insights into how these conditions influence the chemical composition of the samples.

Methods of the instant disclosure can be used to identify low abundance compounds for untargeted analyses of any compound, including chemical, pharmaceutical, biochemical, and metabolomic compounds. Non-limiting examples of chemical, biochemical, and metabolomic compounds include biochemical compounds such as amino acids, peptides and proteins, nucleotides and nucleosides, DNA and RNA fragments, lipids (fatty acids, triglycerides, phospholipids, steroids), carbohydrates (monosaccharides, disaccharides, polysaccharides), vitamins, hormones, and enzymes; metabolomic compounds such as metabolic intermediates (glycolysis intermediates, Krebs cycle intermediates), neurotransmitters, plant metabolites (alkaloids, terpenes, flavonoids), bacterial and fungal metabolites, endogenous metabolites (bile acids, urea cycle intermediates), and xenobiotics (drugs, toxins); environmental contaminants such as pesticides and herbicides, polycyclic aromatic hydrocarbons (PAHs), persistent organic pollutants (POPs), and heavy metals and metalloids (in their organic forms); pharmaceuticals such as therapeutic drugs, drug metabolites, antibiotics; polymeric materials such as polymers and copolymers, additives and plasticizers, and oligomers; isotopically labeled compounds such as stable isotope labeled metabolites, deuterated compounds; industrial chemicals such as dyes and pigments, surfactants and detergents, and explosives; food and beverage analysis such as food additives, flavor compounds, contaminants; forensic analysis such as illicit drugs, explosive residues, and trace evidence compounds; and geological and cosmochemical analysis such as elemental isotopes, and organic molecules in extraterrestrial samples.

While specific types of compounds may be discussed herein, such discussion of specific embodiments is for illustrative purposes and should not be interpreted as limiting the present disclosure to the specific embodiments being illustrated and discussed. In some embodiments, methods of the instant disclosure can be used to detect low-abundance metabolites without the need for isotopologues.

There are multiple categories of chromatography and MS, wherein each combination of chromatography and MS methods can cater to different analytical needs, depending on the complexity of the sample, the required sensitivity, resolution, and speed of analysis. The choice of technique is often determined by the specific application and the nature of the compounds being analyzed.

Non-limiting examples of major categories of chromatographic techniques that can be coupled with MS include Liquid Chromatography-Mass Spectrometry (LC-MS), Gas Chromatography-Mass Spectrometry (GC-MS), Ion Chromatography-Mass Spectrometry (IC-MS), Supercritical Fluid Chromatography-Mass Spectrometry (SFC-MS), Capillary Electrophoresis-Mass Spectrometry (CE-MS). Liquid chromatography methods can include High-Performance Liquid Chromatography (HPLC)—the most common form, utilizing high pressure to pass the sample through a column filled with stationary phase; Ultra-Performance Liquid Chromatography (UPLC)—similar to HPLC but uses smaller particle sizes in the column for higher resolution and faster analysis; Ion Exchange Chromatography-separates ions based on their affinity to an ion exchanger in the column; Size Exclusion Chromatography (SEC)—separates molecules based on size, using a column with pores of a specific size; Normal Phase Chromatography—uses a polar stationary phase and a non-polar mobile phase; Reverse Phase Chromatography—uses a non-polar stationary phase and a polar mobile phase, opposite to normal phase; Chiral Chromatography—separates enantiomers based on their interaction with a chiral stationary phase; Affinity Chromatography—uses a stationary phase made of materials that specifically bind to the analyte of interest. Gas chromatography methods include Capillary Gas Chromatography—uses very narrow capillary tubes with a liquid stationary phase; Packed Column Gas Chromatography—utilizes columns packed with solid stationary phase or solid support coated with liquid stationary phase; Gas-Solid Chromatography (GSC)—involves a solid stationary phase and is used primarily for separating gases or volatile compounds that don't interact with liquid stationary phases. Ion chromatography is typically used for the separation of ions and polar molecules. Supercritical Fluid Chromatography-Mass Spectrometry (SFC-MS) uses a supercritical fluid (like CO2) as the mobile phase, combining aspects of both GC and LC. It's particularly effective for analyzing compounds that are difficult to separate by traditional LC, such as chiral compounds. Capillary Electrophoresis: Although not technically a chromatographic technique, capillary electrophoresis separates ions based on their charge-to-size ratio in an electric field.

Non-limiting examples of MS include Quadrupole Mass Spectrometry (QMS)—uses quadrupole filters for mass analysis, suitable for a broad range of masses; Time-of-Flight Mass Spectrometry (TOF-MS); separates ions by their different flight times; Ion Trap Mass Spectrometry—traps ions using electromagnetic fields and then sequentially ejects them for mass analysis; Fourier Transform Ion Cyclotron Resonance (FT-ICR)—offers very high resolution and accuracy, using a magnetic field to trap ions; Orbitrap Mass Spectrometry uses an electrostatic field to trap ions in an orbital motion around a central electrode; Triple Quadrupole Mass Spectrometry (QqQ) which incorporates three quadrupoles in series; commonly used for quantification due to its high sensitivity and specificity; Tandem Mass Spectrometry (MS/MS) which involves multiple stages of mass spectrometry, often with fragmentation of analyte ions between stages; Quadrupole Time-of-Flight Mass Spectrometry (Q-TOF) which combines quadrupole mass filtering with TOF mass analysis for high accuracy and resolution; and Magnetic Sector Mass Spectrometry which uses a magnetic field to deflect ions, with separation based on mass-to-charge ratio.

In some embodiments, methods of the instant disclosure can be used to overcome drift in retention times of LC-MS runs. In some embodiments, methods of the instant disclosure can be used to overcome drift in retention times of LC-MS runs for analyses of metabolites.

In addition to chromatography, different separation techniques can also be used in conjunction with mass spectrometry. Non-limiting examples of suitable separation techniques other than chromatography include electrophoresis, ion mobility, Field-Flow Fractionation (FFF), Capillary Electrophoresis (CE), Matrix-Assisted Laser Desorption/Ionization (MALDI), Thermal Desorption (TD), Laser Ablation (LA), Desorption Electrospray Ionization (DESI). Electrophoresis separates charged particles under an electric field, effectively used for biomolecules like DNA and proteins. Ion mobility spectrometry, on the other hand, separates ions based on their mobility in a gas phase under an electric field, useful for distinguishing isomers and conformers. FFF techniques separate particles, macromolecules, or colloids based on their size or density in a fluid under various field forces (such as centrifugal, thermal, or electrical fields). Coupling FFF with MS allows for the analysis of large and complex molecules like proteins and polymers. CE is similar to electrophoresis but conducted in capillary tubes. CE is effective for separating ionic species using an electric field. Its coupling with MS provides high resolution and efficiency, particularly useful for analyzing biomolecules like peptides and nucleotides. Although MALDI is more of an ionization technique than a separation technique, it is often mentioned in the context of MS coupling. It's particularly useful for the analysis of large biomolecules like proteins, DNA, and polymers. TD involves heating a sample to release volatile and semi-volatile compounds. When coupled with MS, it allows for the analysis of compounds in air, materials, and environmental samples. LA comprises using a laser to remove material from a solid sample. Coupling LA with MS enables the analysis of solid samples, particularly in fields like geology and material science, allowing for elemental and isotopic analysis. DESI allows for the direct analysis of samples (even from surfaces) under ambient conditions. It's useful for a wide range of applications, including biological tissues and environmental samples.

A method of the instant disclosure comprises calculating a signal bandwidth for identification of low abundance compounds. The signal bandwidth is calculated based on a limit of detection associated with the mass spectrometer and a lowest signal intensity corresponding to an isotopologue associated with the first set of compounds. As used herein a limit of detection refers to the minimum signal intensity that can be distinguished from background noise by a mass spectrometer. These thresholds vary depending on the type and configuration of the mass spectrometer used. For instance, Time-of-Flight (ToF) mass spectrometers, known for their high-speed data acquisition, typically have higher absolute noise thresholds compared to Orbitrap mass spectrometers, which are renowned for their high resolution and sensitivity. ToF instruments might have noise thresholds in the range of a few thousand units, whereas Orbitrap instruments can detect signals as low as a few hundred units due to their superior signal-to-noise ratio. Quadrupole mass spectrometers, often used for targeted analyses, generally exhibit noise thresholds that fall between those of ToF and Orbitrap instruments. The specific noise threshold for any given mass spectrometer is influenced by factors such as the quality of the ion source, the efficiency of ion transmission, and the performance of the detector.

### (b) Method Steps

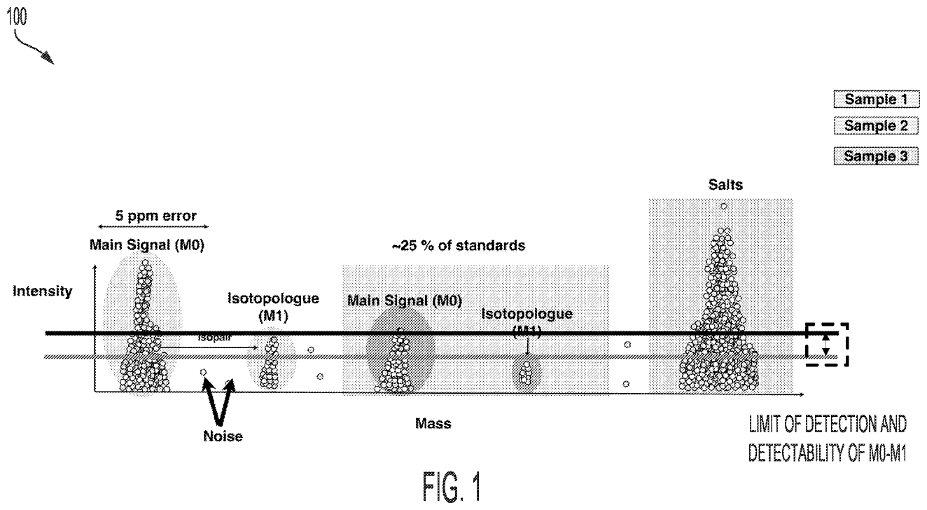

FIG. 1 shows an illustrative example of an environment 100 in which a signal bandwidth within a merged mass spectrum is defined and from which valid metabolite masses can be identified in biological samples without M1 isotopologues in accordance with at least one embodiment. In the environment 100, samples taken from a living system (e.g., human, etc.) may include multiple unique metabolites and inorganic salts. Some of these unique metabolites may be faint within the obtained samples but are nevertheless biologically important such that detection and classification of these metabolites are needed for different clinical applications. In an embodiment when chromatography-MS used is liquid chromatography-MS (LC-MS), different samples taken from a living system are processed by an LC-MS machine to identify sets of signals corresponding to the different metabolites and inorganic salts. Different metabolites within these samples can be identified in each spectrum obtained from a sample. In some embodiments, these sets of signals from the different samples can be pooled together in a merged mass spectrum for identification of different metabolites within these samples.

The merged mass spectrum may result in more signal intensity measurements representing true masses hyper-concentrating along Gaussian (or other non-random) distributions (e.g., within 5 parts per million (ppm)) corresponding to the masses of different compounds. The concentration of signals may be increased from a signal/noise ratio without requiring signal filtering. In an embodiment, compounds of interest within the merged mass spectrum may include metabolites or other types of organic compounds. An isotopologue of a compound may include, for example, a carbon-13 isotope atom. While such isotopes may naturally occur, such occurrence may be at relatively low levels (e.g., 1% of abundance relative to the associated compound). A true signal for a specific compound may therefore be accompanied by a valid signal of a naturally-occurring isotopologue that is lower in abundance and whose signal is offset from the true signal by exactly the mass difference between the dominant and rarer isotopic species of an element and its (e.g., carbon-13) atom(s). Similar to how aggregated compound signals may hyper-concentrate around the mass of the compound, aggregated isotopologue signals may similarly hyper-concentrate around the mass of the isotopologue. Thus, when m/z-signal intensities from multiple samples are combined into a single, merged file (e.g., a merged mass spectrum) of m/z signal-intensities, the probability of finding a pair of signals offset by the exact mass of one or more carbon-13 atoms (representing a compound and its naturally-occurring isotopologue(s)) at a single retention time window increases significantly. (The chromatogram data can be included in the file of m/z signal intensities if available to ensure that compounds and their isotopologues elute at similar retention times.) An isotopologue can then be detected where the merged file of m/z signal-intensities contains two peaks offset by one or more 13C atoms. Sets of signals linked by a mass shift that is an integer multiple of the mass of a 13C atom may be referred to herein as “isopairs” and may occur at the same retention time as the parent metabolite, although these can be detected by mass shift alone if other dimensions of separation are not available. The presence of the isotopologue peak may further increase confidence in the determination that the associated compound peak is actually associated with the compound (e.g., rather than noise or any inorganic salts).

In many instances, while the aforementioned process may be used to identify isopairs from a merged mass spectrum within a detection threshold (e.g., the threshold at which isotopologues corresponding to a compound can be detected), metabolites that are below this detection threshold may be more difficult to detect. For instance, metabolites (especially with a lower number of carbon atoms (thereby allowing fewer opportunities for assimilation of 13C atoms)) at levels of approximately 50,000 units across all samples in a dataset may be undetectable through the aforementioned process as the isotopologues corresponding to these metabolites may be less than 1,000-2,000 units across all samples in the dataset. Thus, these isotopologues may be below the limit of detection.

In an embodiment, valid metabolite masses without isopairs are confined to a limited bandwidth defined as being between the absolute noise threshold (e.g., approximately 1,000 units across spectrometers) and 200,000 units. This limited bandwidth, in some instances, may be determined based on an evaluation of different sample datasets for which statistical data corresponding to metabolite presence within these different sample datasets and their corresponding signal intensities is available. Focusing on this limited bandwidth within a merged mass spectrum may allow for the immediate removal of up to 90 percent of signals (including noise signals) while the expected abundance of metabolites and salts may range up to 10\hat{ }9 units or greater. Thus, the identification of an ideal signal bandwidth from which valid metabolite masses may be identified without corresponding isotopologues may limit noise detection by immediately constraining the potential for false positive classification of inorganic salts as metabolites since these inorganic salts may frequently have signals above this ideal signal bandwidth.

In an embodiment, the ceiling for this ideal signal bandwidth is dynamically calculated by identifying the signal intensity corresponding to the least abundant metabolite that is part of a valid isopair. As noted above, when m/z-signal intensities from multiple samples are combined into a merged mass spectrum of m/z signal-intensities, the probability of finding a pair of signals offset by the exact mass of one or more carbon-13 atoms (representing a compound and its naturally-occurring isotopologue(s)) at a single retention time window increases significantly. Through this process, valid isopairs may be identified from the merged mass spectrum. From these valid isopairs, the least abundant metabolite having a corresponding isotopologue may be identified. The signal intensity of this least abundant metabolite may be identified from the merged mass spectrum and used to define the ceiling for the ideal signal bandwidth that is to be used to identify metabolites from the merged mass spectrum without requiring isotopologues for these metabolites. This ceiling for metabolite detection without an isotopologue can also be verified via theoretical calculations by multiplying the limit of detection by natural abundance of the isotopologue and the lowest number of atoms in a metabolite molecule which could conceivably be comprised by this isotopologue.

As illustrated in FIG. 1, the ideal signal bandwidth is calculated with a lower signal limit corresponding to the limit of detection for the LC-MS. Further, this ideal signal bandwidth may have an upper signal limit corresponding to the peak of the isotopologue corresponding to the least abundant metabolite (e.g., isopair) within the merged mass spectrum. By calculating the upper signal limit according to the detection of isopairs from the merged mass spectrum, the signal bandwidth for detecting metabolites without corresponding isotopologues may be defined. Further, because the limit of detection for the LC-MS is above the absolute noise threshold (e.g., approximately 1,000 units across spectrometers), this ideal signal bandwidth may automatically remove a significant amount of noise signals from the merged mass spectrum, thereby reducing the likelihood of noise signals being classified as metabolites. Additionally, inorganic salts, by virtue of having signal intensities above the upper signal limit corresponding to the peak of the least abundant metabolite having a valid isopair, may be automatically filtered out from the merged mass spectrum when evaluating the merged mass spectrum within this calculated ideal signal bandwidth.

Within the calculated ideal signal bandwidth, the merged mass spectrum may include an abundance of both signals corresponding to metabolites without associated isotopologues (as their corresponding isotopologues may be below the limit of detection) and electrostatic noise signals that rise stochastically above the limit of detection. In an embodiment, regions of noise within the ideal signal bandwidth can be distinguished from those corresponding to valid metabolites based on certain characteristics associated with the merged mass spectrum. For instance, the merged mass spectrum may accentuate the innate statistical differences of signals corresponding to valid metabolites and signals corresponding to electrostatic noise. Signals corresponding to valid metabolites within the merged mass spectrum, for example, may be highly non-random, whereas signals corresponding to electrostatic noise may be far more random in nature.

In an embodiment, within the calculated ideal signal bandwidth, thin regions (e.g., bins) of the merged mass spectrum (e.g., 1/10,000th Daltons) are often adjacently occupied only by signals corresponding to valid, but otherwise faint, metabolites. While electrostatic noise signals may be statistically biased (e.g., the electrostatic noise signals may congregate in regions within the merged mass spectrum), these electrostatic noise signals may still be sufficiently sparse such that it is statistically improbable for two noise signals to occupy adjacent bins that are 1/10,000th Daltons wide. Thus, through an evaluation of these bins within the ideal signal bandwidth, metabolites and noise signals may be classified according to any detected signal adjacencies amongst these bins.

The calculation of an ideal signal bandwidth for the classification of metabolites within a merged mass spectrum without requiring corresponding isotopologues can provide further advantages. For instance, in an embodiment, regions above the calculated ideal signal bandwidth are generally divided further into thinner bins (e.g., 1/100,000th Daltons wide) during isopair calculation. However, by utilizing the merged mass spectrum region corresponding to the calculated ideal signal bandwidth, the corresponding bins can be extended up to 10 times or more (e.g., from 1/100,000th Daltons to 1/10,000th Daltons) to increase the sensitivity to faint metabolites within the merged mass spectrum without picking up a correspondingly large amount of electrostatic noise signals. Further, in an embodiment, adjacently filled bins of the merged mass spectrum can be identified within the calculated ideal signal bandwidth by mapping signals to adjacent regions with a mass shift of 0.0001 Daltons. This may allow for the rapid detection of metabolite masses within the calculated ideal signal bandwidth without requiring corresponding isotopologues.

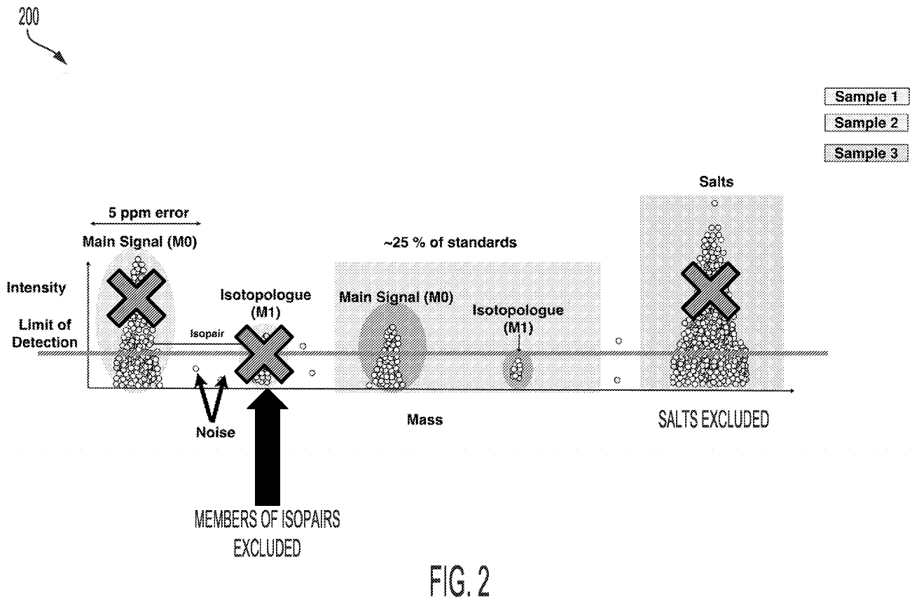

FIG. 2 shows an illustrative example of an environment 200 in which metabolites with M0-M1 pairs and inorganic salts are filtered out to allow for identification of metabolite masses without M1 isotopologues in accordance with at least one embodiment. In the environment 200, the calculation of an ideal signal bandwidth for the identification of faint metabolites within a merged mass spectrum may allow for the exclusion, or filtering, of identified and valid isopairs and inorganic salts from the merged mass spectrum. As noted above, when m/z-signal intensities from multiple samples are combined into a merged mass spectrum of m/z signal-intensities, the probability of finding a pair of signals offset by the exact mass of one or more carbon-13 atoms (representing a compound and its naturally-occurring isotopologue(s)) at a single retention time window increases significantly. From this merged mass spectrum, an isotopologue can then be detected where the merged mass spectrum contains two peaks offset by one or more 13C atoms. Thus, through this process, valid isopairs corresponding to different metabolites may be identified.

In an embodiment, when the ideal signal bandwidth for detecting faint metabolites from the merged mass spectrum is calculated, any previously identified isopairs may be excluded from the merged mass spectrum within the ideal signal bandwidth. As noted above, the ceiling for the ideal signal bandwidth may be calculated by identifying the signal intensity corresponding to the least abundant metabolite that is part of a valid isopair. For example, the signal intensity corresponding to the ceiling of the ideal signal bandwidth may be calculated according to the peak signal intensity of the isotopologue corresponding to the least abundant metabolite identified according to the aforementioned process of finding pairs of signals offset by the exact mass of one or more carbon-13 atoms. This process (hereinafter referred to as an “autocredential process”) may allow for the identification of any isopairs for which corresponding isotopologues have signal intensities above the limit of detection for the LC-MS.

In addition to valid isopairs, any inorganic salts may be automatically filtered out from the merged mass spectrum within the ideal signal bandwidth calculated based on the limit of detection and the signal intensity of the least abundant metabolite associated with a detected isopair. For instance, inorganic salts (as illustrated in FIG. 2) may have signal intensities that greatly exceed the peak signal intensity of the isotopologue corresponding to the least abundant metabolite for which a valid isopair is detected. Accordingly, when the ideal signal bandwidth for identification of faint metabolites without corresponding isotopologues is calculated, the inorganic salts within the merged mass spectrum may be automatically excluded as a result of their corresponding signal intensities exceeding the ceiling of the calculated ideal signal bandwidth.

As illustrated in FIG. 2, the remaining signal intensities above the limit of detection and that may be evaluated within the ideal signal bandwidth may include any faint metabolites for which corresponding isotopologues have signals below the limit of detection and any electrostatic noise signals that rise stochastically above the limit of detection. This may reduce the amount of signals that need to be evaluated within the calculated ideal signal bandwidth, thereby reducing the likelihood of false positive classification of noise signals and inorganic salts as metabolites while also reducing the amount of processing required to identify valid metabolites within this ideal signal bandwidth without the need for detecting corresponding isotopologues.

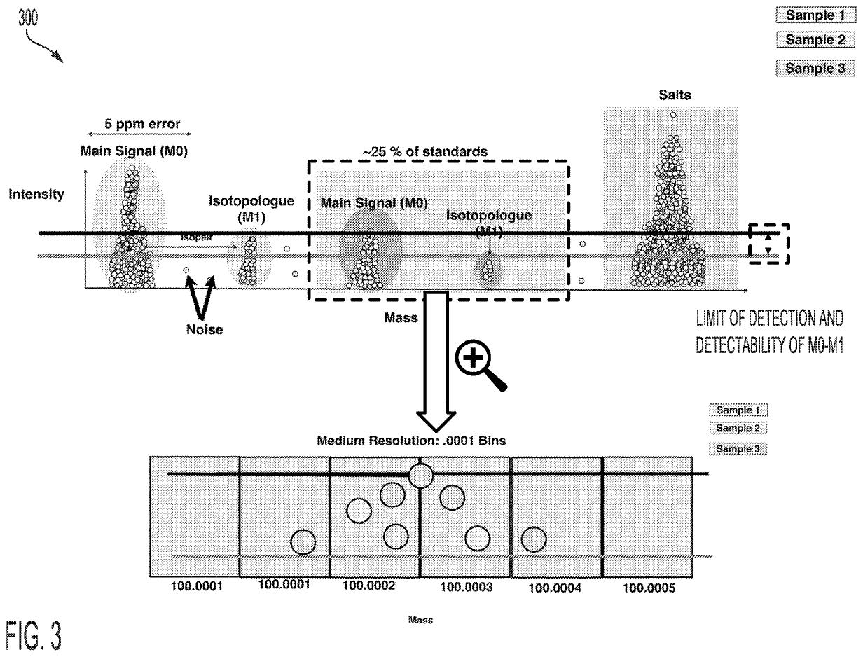

FIG. 3 shows an illustrative example of an environment 300 in which signal distributions within a defined signal bandwidth are evaluated to identify metabolite masses without M1 isotopologues in accordance with at least one embodiment. In the environment 300, a merged mass spectrum comprising multiple samples is evaluated within a calculated signal bandwidth to identify any faint metabolites present within the merged mass spectrum for which corresponding isotopologues are undetectable (e.g., have signal intensities below the limit of detection for the LC-MS). As noted above, through an autocredential process, valid isopairs may be identified from the merged mass spectrum. These valid isopairs may be excluded from the merged mass spectrum within the calculated signal bandwidth to allow for identification of faint metabolites within this calculated signal bandwidth. Further, as inorganic salts may have signal intensities greatly exceeding the ceiling of this calculated signal bandwidth, these inorganic salts may be immediately identified and excluded from the merged mass spectrum for this calculated signal bandwidth.

In an embodiment, and as illustrated in FIG. 3, within the calculated signal bandwidth, the merged mass spectrum can be divided into thin regions, or bins, that can be evaluated to identify any signal adjacencies that may denote the presence of a faint metabolite within the merged mass spectrum. For example, as illustrated in FIG. 3, the merged mass spectrum within the calculated signal bandwidth is dividing into bins corresponding 1/10,000th Dalton mass ranges. Through evaluation of these bins, a system may identify any adjacently occupied bins. For instance, as illustrated in FIG. 3, the merged mass spectrum may include adjacently occupied bins corresponding to masses of 100.0001 Daltons to 100.0004 Daltons, with an associated signal peak at the calculated signal bandwidth ceiling and corresponding to a mass between 100.0002 and 100.0003 Daltons. These adjacently occupied bins may be indicative of a faint metabolite for which a corresponding isotopologue is undetectable.

Through this process of dividing the merged mass spectrum into individual bins within the calculated signal bandwidth, any electrostatic noise signals may be classified as such through statistical analysis. For instance, while electrostatic noise signals may be statistically biased (e.g., the electrostatic noise signals may congregate in regions within the merged mass spectrum), these electrostatic noise signals may still be sufficiently sparse such that it is statistically improbable for two noise signals to occupy adjacent bins that are 1/10,000th Daltons wide. Thus, through an evaluation of these bins within the calculated signal bandwidth, metabolites and noise signals may be classified according to any detected signal adjacencies amongst these bins. This is because noise signals will not have signal adjacencies, whereas metabolites (whose signals are non-random) will.

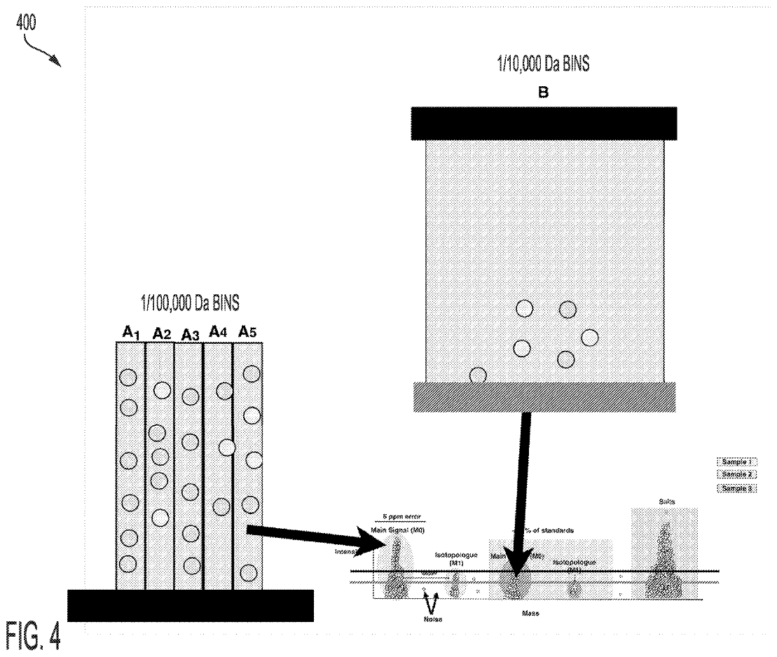

FIG. 4 shows an illustrative example of an environment 400 in which adjacently filled bins of the merged mass spectrum are identified through mapping signals to adjacent regions with a particular mass shift in accordance with at least one embodiment. In the environment 400, once an ideal signal bandwidth has been calculated for the identification of faint metabolites within a merged mass spectrum, the merged mass spectrum above the upper limit or ceiling of the ideal signal bandwidth may be divided into thin regions, or bins, through which isotopologues may be identified according to the adjacency of corresponding signals within these bins. For instance, as illustrated in FIG. 4, the merged mass spectrum above the upper limit or ceiling of the ideal signal bandwidth may be divided into 1/100,000th Dalton wide bins. From these bins, above the upper limit or ceiling of the ideal signal bandwidth, isotopologues corresponding to valid isopairs within the merged mass spectrum may be identified. For instance, signal adjacencies amongst these bins may statistically represent a metabolite or isotopologue as opposed to electrostatic noise within the merged mass spectrum, as described in greater detail herein.

In an embodiment, the bins within the ideal signal bandwidth can be extended up to ten times the size of the bins above the upper limit or ceiling of the ideal signal bandwidth to increase the sensitivity to faint metabolites within the merged mass spectrum without picking up a correspondingly large quantity of electrostatic noise. For example, as illustrated in the environment 400, the bins within the ideal signal bandwidth can be implemented to be ten times larger than the bins generated above the upper limit of ceiling of the ideal signal bandwidth (e.g., 1/10,000th Daltons within the ideal signal bandwidth compared to 1/100,000th Daltons above the ideal signal bandwidth).

As noted above, the merged mass spectrum accentuates the innate statistical differences of signals from metabolites (e.g., highly non-random) and of electrostatic noise signals (e.g., more random). Thus, while the bins within the ideal signal bandwidth are extended up to ten times the size of the bins above the upper limit or ceiling of the ideal signal bandwidth, the probability of a false positive classification of a metabolite within the ideal signal bandwidth is exceedingly low. For instance, as illustrated in FIG. 4, the total signal within the 1/10,000th Dalton bin inside of the ideal signal bandwidth may be approximately equal to the total signal within each of the 1/100,000th bins above the upper limit or ceiling of the ideal signal bandwidth and corresponding to a known valid metabolite within the merged mass spectrum. Accordingly, the probability of the signal within the illustrated 1/10,000th Dalton bin within the ideal signal bandwidth being a metabolite as opposed to an electrostatic noise signal is considered high such that the signal may be classified as a metabolite with a high degree of confidence.

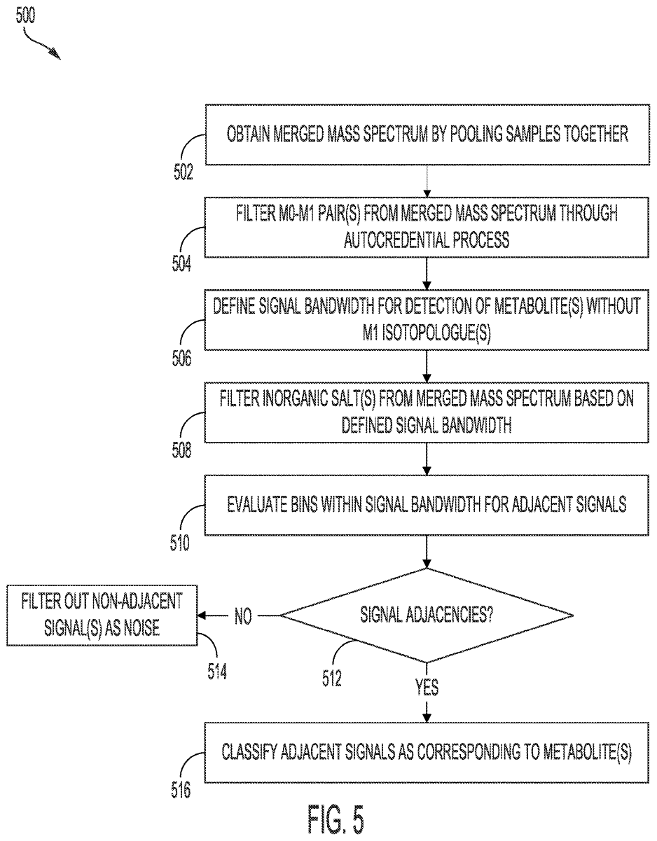

FIG. 5 shows an illustrative example of a process 500 for evaluating bins within a merged mass spectrum to identify metabolite masses without requiring detection of corresponding M1 isotopologues in accordance with at least one embodiment. The process 500 may be performed by a system configured to evaluate a merged mass spectrum constructed from a dataset that includes multiple samples. At step 502, the system may obtain a merged mass spectrum by pooling a plurality of samples together. The merged mass spectrum, as described above, may amplify signals within the various samples such that normally undetectable M1 isotopologue peaks that are above the limit of detection for the LC-MS emerge. Further, faint metabolites that lack a detectable M1 isotopologue may also have their M0 signal peaks amplified through the pooling of samples in the dataset to form the merged mass spectrum.

At step 504, the system may filter out any M0-M1 pairs (e.g., isopairs) corresponding to valid metabolites from the merged mass spectrum through an autocredential process. As noted above, when multiple samples are combined into a merged mass spectrum, the probability of finding a pair of signals offset by the exact mass of one or more carbon-13 atoms at a single retention time window increases significantly. From this merged mass spectrum, an isotopologue can then be detected where the merged mass spectrum contains two peaks offset by one or more 13C atoms. Through this autocredential process, valid isopairs corresponding to different metabolites may be identified. As the purpose of the process 500 is to identify any metabolites present in the merged mass spectrum that may not have detectable isotopologues, the valid isopairs identified through the autocredential process may not be required outside of defining the ideal signal bandwidth for detecting the faint metabolites within the merged mass spectrum, as described in greater detail herein. Thus, these valid isopairs may be filtered out of the merged mass spectrum in order to allow detection of the second set of metabolites, which are those which reside in the previously described signal bandwidth and whose isotopologues are undetectable.

At step 506, the system may define an ideal signal bandwidth within which faint metabolites may be identified without requiring identification of corresponding isotopologues that may be below the limit of detection of the LC-MS. To define the ideal signal bandwidth for identification of these faint isotopologues, the system may determine the limit of detection for the LC-MS. This limit of detection may be pre-defined based on the characteristics of the one of more LC-MS systems from which the samples were obtained. This limit of detection may serve as the lower limit or floor for the ideal signal bandwidth to be used to detect faint metabolites from the merged mass spectrum.

To determine the upper limit or ceiling of the ideal signal bandwidth, the system may evaluate the previously identified isopairs to identify the signal intensity corresponding to the least abundant metabolite that is associated with a valid isopair. For example, the system may identify, from the set of isopairs identified during the autocredential process, the peak signal intensity of an isotopologue associated with a valid isopair and for which its corresponding metabolite is identified as being the least abundant within the set of valid isopairs. Accordingly, this peak signal intensity of the isotopologue associated with this valid isopair may be used as the upper limit or ceiling of the ideal signal bandwidth for identifying any metabolites for which corresponding isotopologues may be undetectable.

Once the system has defined the signal bandwidth for identifying any faint metabolites within the merged mass spectrum without requiring identification of their corresponding isotopologues, the system, at step 508, may filter out any inorganic salts from the merged mass spectrum. As noted above, inorganic salts may have signal intensities that greatly exceed the peak signal intensity of the isotopologue corresponding to the least abundant metabolite for which a valid isopair is detected. Accordingly, when the ideal signal bandwidth for identification of faint metabolites without corresponding isotopologues is calculated, the inorganic salts within the merged mass spectrum may be automatically excluded as a result of their corresponding signal intensities exceeding the ceiling of the calculated ideal signal bandwidth.

At step 510, the system may evaluate the various bins within the defined ideal signal bandwidth for any adjacent signals that may be indicative of the presence of faint metabolites within the ideal signal bandwidth. For instance, the system may divide the merged mass spectrum, within this defined signal bandwidth, into thin regions (e.g., bins) having widths corresponding to 1/10,000th Dalton mass ranges. Signals within these bins may correspond to either faint metabolites present within the merged mass spectrum or to electrostatic noise signals which rise stochastically above the limit of detection. The width of these bins may be determined through statistical analysis of previously processed sample datasets and corresponding identification of metabolites, inorganic salts, and electrostatic noise signals from these sample datasets. For instance, the width of the bins may be determined based on the innate statistical differences of signals associated with metabolites (e.g., highly non-random) and of signals associated with electrostatic noise (e.g., more random). Thus, based on the highly non-random nature of metabolite signals within merged mass spectra, the system may define the width of the bins such that these metabolite signals may be detected through signal adjacencies within these bins, while noise signals will be highly unlikely to occupy adjacent bins. Further, while electrostatic noise signals may be statistically biased (e.g., congregate in regions of the merged mass spectrum), these electrostatic noise signal clusters may be sufficiently sparse such that the likelihood of electrostatic noise signals being adjacent within the defined bins is exceedingly low.

At step 512, the system may determine whether there are any signal adjacencies within the bins defined within the calculated signal bandwidth. For instance, the system may evaluate the defined bins to identify any signal adjacencies that may be indicative of the presence of a faint metabolite within the merged mass spectrum. For any signals that have no adjacencies within the merged mass spectrum, the system, at step 514, may filter out these signals as being likely electrostatic noise within the merged mass spectrum and not being associated with a metabolite. However, for any signals that are statistically adjacent according to the defined bins, the system, at step 516, may classify these signals as corresponding to a faint metabolite. Thus, through the process 500, the system may process a merged mass spectrum to identify any faint metabolites that may be associated with isotopologues that are otherwise undetectable from the merged mass spectrum. Accordingly, metabolites may be identified from a merged mass spectrum without requiring identification of corresponding isotopologues.

As described herein above, the disclosed technique is highly scalable and has been successfully implemented on large datasets, demonstrating efficacy in identifying metabolites that were previously undetectable using conventional methods. More specifically, the methods of the instant disclosure were used with samples obtained from 700 COVID-19 patients and were analyzed using Time-of-Flight Mass Spectrometry (ToF MS). ToF MS can have a sufficiently fast scan rate to exclude isotopologues, providing an additional reason why it is necessary to identify compounds without relying on isotopologues. This necessity arises not just because isotopologues may be below the threshold of detection, but also due to the rapid scan rates of ToF MS. The samples were previously determined to comprise 130 known metabolites, identified using compound standards. However, using standards for compound identification in a sample has many drawbacks. By employing the methods of the instant disclosure, 125 of the 130 known compounds in the samples were identified in an untargeted manner, without the use of isotopologues or standards. Importantly, for comparison, when the compounds were identified using methods that rely on isotopologues, only 55 out of the known 130 metabolites were identified. This highlights the superior efficacy of the disclosed methods in identifying low-abundance metabolites without the need for isotopologues or standards.

### (c) Embodiments

One aspect of the present disclosure encompasses a computer-implemented method. The computer-implemented method comprises receiving a plurality of samples. The plurality of samples includes mass-to-charge (m/z) signal intensities captured by a mass spectrometer. Further, the m/z signal intensities correspond to signals associated with mass measurements of metabolites within the plurality of samples. The computer-implemented method further comprises combining the plurality of samples into a merged mass spectrum of the m/z signal intensities. The computer-implemented method further comprises identifying a first set of metabolites from the merged mass spectrum. The first set of metabolites is identified based on a concentration of signals corresponding to mass measurements associated with the first set of metabolites and isotopologues of the first set of metabolites. The computer-implemented method further comprises calculating a signal bandwidth for identification of a second set of metabolites. The signal bandwidth is calculated based on a lower bound which is the limit of detection associated with the mass spectrometer's innate sensitivity and bounded above by the a lowest signal intensity corresponding to an isotopologue associated with the first set of metabolites. The computer-implemented method further comprises identifying a set of signal intensity adjacencies within this signal bandwidth. The set of signal intensity adjacencies are indicative of a second set of metabolites.

In some embodiments, the method can further comprise receiving or having received the plurality of chromatography-MS mass spectra. In some embodiments, the computer-implemented method further comprises dividing the merged mass spectrum within the signal bandwidth into bins. The bins can be sized to detect the set of signal intensity adjacencies within the signal bandwidth. In some embodiments, the bins correspond to an atomic mass range equivalent to 1/10,000th Daltons.

In some embodiments, the computer-implemented method further comprises identifying one or more inorganic salts from the merged mass spectrum. The one or more inorganic salts are identified as a result of signal intensities corresponding to the one or more inorganic salts exceeding the lowest signal intensity required to have an isotopologue (but lacking one because it does not contain carbon), while also having a signal peak outside of the described intensity bandwidth.

In some embodiments, the set of signal intensity adjacencies are identified according to statistical differences between known metabolite signal intensities and electrostatic noise signal intensities.

In some embodiments, the computer-implemented method further comprises filtering the first set of metabolites from the merged mass spectrum prior to identifying the set of signal intensity adjacencies within the signal bandwidth.

In some embodiments, the second set of metabolites is identified without requiring identification of corresponding isotopologues from the merged mass spectrum.

Another aspect of the instant disclosure encompasses a method for detecting compounds with weak signals in mass spectrometry (MS) data. The method comprises (a) combining or having combined a plurality of chromatography-mass spectrometry (chromatography-MS) mass spectra comprising mass-to-charge (m/z) signal intensities associated with mass measurements of compounds into a merged mass spectrum of m/z signal intensities; (b) identifying a first set of compounds from the merged mass spectrum, wherein the first set of compounds is identified based on a concentration of signals corresponding to mass measurements associated with the first set of compounds and isotopologues of the first set of compounds; (c) calculating a signal bandwidth for identification of a second set of compounds, wherein the signal bandwidth is calculated based on a limit of detection associated with the mass spectrometer and a lowest signal intensity corresponding to an isotopologue associated with the first set of compounds; and (d) identifying a set of signal intensity adjacencies within the signal bandwidth, wherein the set of signal intensity adjacencies are indicative of a second set of compounds. The method can optionally comprise filtering signals associated with the first set of compounds from the merged mass spectrum prior to identifying the set of signal intensity adjacencies within the signal bandwidth. The method can also optionally comprise iteratively repeating steps (b) to (d). Importantly, the second set of compounds can be identified without requiring identification of corresponding isotopologues from the merged mass spectrum.

In some embodiments, the method further comprises receiving or having received the plurality of chromatography-MS mass spectra. In some embodiments, the compound is metabolites, isomers of metabolites, or both. Additionally, the chromatography-MS can be liquid chromatography-MS (LC-MS).

In some embodiments, a method of the instant disclosure further comprises dividing the merged mass spectrum within the signal bandwidth into bins, wherein the bins are sized to detect the set of signal intensity adjacencies within the signal bandwidth. In some embodiments, the set of signal intensity adjacencies are identified according to statistical differences between known metabolite signal intensities and electrostatic noise signal intensities. In some embodiments, the bins correspond to an atomic mass range equivalent to 1/10,000th Daltons.

The method can also further comprise identifying one or more inorganic salts from the merged mass spectrum, wherein the one or more inorganic salts are identified as a result of signal intensities corresponding to the one or more inorganic salts exceeding a required lowest signal intensity corresponding to an expected isotopologue, while there is no expected isotopologue detected.

In some embodiments, the set of signal intensity adjacencies are identified according to statistical differences between known metabolite signal intensities and electrostatic noise signal intensities. Further, the second set of compounds can be identified without requiring identification of corresponding isotopologues from the merged mass spectrum.

### II. Computing System

Another embodiment of the instant disclosure encompasses a computing system for implementing a method of the instant disclosure. In some embodiments, the computing system is attached to and/or used in conjunction with a chromatography-MS system. The computing system can comprise several known components and circuitry, including a processor, a memory system, input and output devices and interfaces (e.g., an interconnection mechanism), as well as other components, such as transport circuitry (e.g., one or more busses), a video and audio data input/output (I/O) subsystem, special-purpose hardware, as well as other components and circuitry, as described below in more detail. Further, the computer system(s) can be a multi-processor computer system or may include multiple computers connected over a computer network.

A processor can include one or more general purpose computers, dedicated microprocessors, graphics processors, or other processing devices capable of communicating electronic information. Non-limiting examples of a processor include one or more application-specific integrated circuits (ASICs), graphical processing units (GPUs), field programmable gate arrays (FPGAs), graphics processing units (GPUs), digital signal processors (DSPs) and any other suitable specific or general purpose processors. The processor can be implemented as appropriate in hardware, firmware, or combinations thereof with computer-executable instructions and/or software. Computer-executable instructions and software can include computer-executable or machine-executable instructions written in any suitable programming language to perform the various functions described.

The memory can include more than one memory and can be distributed throughout the computing system. The memory can store program instructions that are loadable and executable on the processor(s) as well as data generated during the execution of these programs. Depending on the configuration and type of memory, the memory can be volatile (such as random access memory (RAM)) and/or non-volatile (such as read-only memory (ROM), flash memory, or other memory). In some embodiments, the memory can include multiple different types of memory, such as static random access memory (SRAM), dynamic random access memory (DRAM), or ROM.

In some embodiments, the computing system can also include additional storage, which can include removable storage and/or non-removable storage. The additional storage can include, but is not limited to, magnetic storage, optical disks, and/or solid-state storage. The disk drives and their associated computer-readable media can provide non-volatile storage of computer-readable instructions, data structures, program modules, and other data for the computing devices. The memory and the additional storage, both removable and non-removable, are examples of computer-readable storage media. For example, computer-readable storage media can include volatile or non-volatile, removable, or non-removable media implemented in any suitable method or technology for storage of information such as computer-readable instructions, data structures, program modules, or other data. As used herein, modules, engines, and components, can refer to programming modules executed by computing systems (e.g., processors) that are part of the architecture.

The processor generally manipulates the data within the integrated circuit memory element in accordance with the program instructions and then copies the manipulated data to the non-volatile recording medium after processing is completed. A variety of mechanisms are known for managing data movement between the non-volatile recording medium and the integrated circuit memory element, and the computing system that implements the methods, steps, systems control and system elements control described above is not limited thereto. The computing system is not limited to a particular memory system.

At least part of such a memory system described above can be used to store one or more data structures (e.g., look-up tables) or equations such as calibration curve equations. For example, at least part of the non-volatile recording medium can store at least part of a database that includes one or more of such data structures. Such a database can be any of a variety of types of databases, for example, a file system including one or more flat-file data structures where data is organized into data units separated by delimiters, a relational database where data is organized into data units stored in tables, an object-oriented database where data is organized into data units stored as objects, another type of database, or any combination thereof.

The computer implemented control system(s) can include one or more output devices. Non-limiting example output devices include a cathode ray tube (CRT) display, liquid crystal displays (LCD) and other video output devices, printers, communication devices such as a modem or network interface, storage devices such as disk or tape, and audio output devices such as a speaker.

The computing system also can include one or more input devices. Example input devices include a keyboard, keypad, track ball, mouse, pen and tablet, communication devices such as described above, and data input devices such as audio and video capture devices and sensors. The computing system is not limited to the particular input or output devices described herein.

It should be appreciated that one or more of any type of computing system can be used to implement various embodiments described herein. Aspects of the invention may be implemented in software, hardware or firmware, or any combination thereof. The computing system can include specially programmed, special purpose hardware, for example, an application-specific integrated circuit (ASIC). Such special-purpose hardware can be configured to implement one or more of the methods, steps, simulations, algorithms, systems control, and system elements control described above as part of the computer implemented control system(s) described above or as an independent component.

The computing system and components thereof may be programmable using any of a variety of one or more suitable computer programming languages. Such languages may include procedural programming languages, for example, LabView, C, Pascal, Fortran and BASIC, object-oriented languages, for example, C++, Java and Eiffel and other languages, such as a scripting language or even assembly language.

The methods, steps, simulations, algorithms, systems control, and system elements control may be implemented using any of a variety of suitable programming languages, including procedural programming languages, object-oriented programming languages, other languages and combinations thereof, which may be executed by such a computer system. Such methods, steps, simulations, algorithms, systems control, and system elements control can be implemented as separate modules of a computer program, or can be implemented individually as separate computer programs. Such modules and programs can be executed on separate computers.

Such methods, steps, simulations, algorithms, systems control, and system elements control, either individually or in combination, may be implemented as a computer program product tangibly embodied as computer-readable signals on a computer-readable medium, for example, a non-volatile recording medium, an integrated circuit memory element, or a combination thereof. For each such method, step, simulation, algorithm, system control, or system element control, such a computer program product may comprise computer-readable signals tangibly embodied on the computer-readable medium that define instructions, for example, as part of one or more programs, that, as a result of being executed by a computer, instruct the computer to perform the method, step, simulation, algorithm, system control, or system element control.

For clarity of explanation, in some instances the present technology may be presented as including individual functional blocks including functional blocks comprising devices, device components, steps or routines in a method embodied in software, or combinations of hardware and software.

Any of the steps, operations, functions, or processes described herein may be performed or implemented by a combination of hardware and software services or services, alone or in combination with other devices. In some embodiments, a service can be software that resides in memory of a client device and/or one or more servers of a content management system and perform one or more functions when a processor executes the software associated with the service. In some embodiments, a service is a program, or a collection of programs that carry out a specific function. In some embodiments, a service can be considered a server. The memory can be a non-transitory computer-readable medium.

In some embodiments the computer-readable storage devices, mediums, and memories can include a cable or wireless signal containing a bit stream and the like. However, when mentioned, non-transitory computer-readable storage media expressly exclude media such as energy, carrier signals, electromagnetic waves, and signals per se.

Methods according to the above-described examples can be implemented using computer-executable instructions that are stored or otherwise available from computer readable media. Such instructions can comprise, for example, instructions and data which cause or otherwise configure a general purpose computer, special purpose computer, or special purpose processing device to perform a certain function or group of functions. Portions of computer resources used can be accessible over a network. The computer executable instructions may be, for example, binaries, intermediate format instructions such as assembly language, firmware, or source code. Examples of computer-readable media that may be used to store instructions, information used, and/or information created during methods according to described examples include magnetic or optical disks, solid state memory devices, flash memory, USB devices provided with non-volatile memory, networked storage devices, and so on.

Devices implementing methods according to these disclosures can comprise hardware, firmware and/or software, and can take any of a variety of form factors. Typical examples of such form factors include servers, laptops, smart phones, small form factor personal computers, personal digital assistants, and so on. Functionality described herein also can be embodied in peripherals or add-in cards. Such functionality can also be implemented on a circuit board among different chips or different processes executing in a single device, by way of further example.

The instructions, media for conveying such instructions, computing resources for executing them, and other structures for supporting such computing resources are means for providing the functions described in these disclosures.

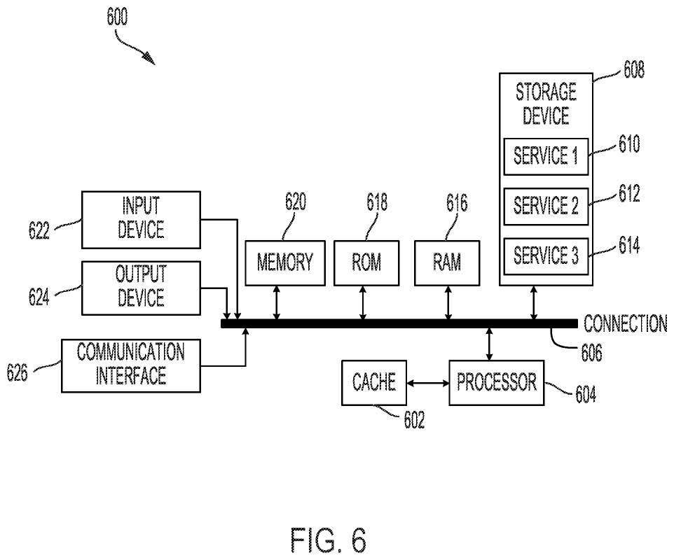

FIG. 6 illustrates a computing system architecture 600 including various components in electrical communication with each other using a connection 606, such as a bus, in accordance with some implementations. Example system architecture 600 includes a processing unit (CPU or processor) 604 and a system connection 606 that couples various system components including the system memory 620, such as ROM 618 and RAM 616, to the processor 604. The system architecture 600 can include a cache 602 of high-speed memory connected directly with, in close proximity to, or integrated as part of the processor 604. The system architecture 600 can copy data from the memory 620 and/or the storage device 608 to the cache 602 for quick access by the processor 604. In this way, the cache can provide a performance boost that avoids processor 604 delays while waiting for data. These and other modules can control or be configured to control the processor 604 to perform various actions.

Other system memory 620 may be available for use as well. The memory 620 can include multiple different types of memory with different performance characteristics. The processor 604 can include any general purpose processor and a hardware or software service, such as service 1 610, service 2 612, and service 3 614 stored in storage device 608, configured to control the processor 604 as well as a special-purpose processor where software instructions are incorporated into the actual processor design. The processor 604 may be a completely self-contained computing system, containing multiple cores or processors, a bus, memory controller, cache, etc. A multi-core processor may be symmetric or asymmetric.

To enable user interaction with the computing system architecture 600, an input device 622 can represent any number of input mechanisms, such as a microphone for speech, a touch-sensitive screen for gesture or graphical input, keyboard, mouse, motion input, speech and so forth. An output device 624 can also be one or more of a number of output mechanisms known to those of skill in the art. In some instances, multimodal systems can enable a user to provide multiple types of input to communicate with the computing system architecture 600. The communications interface 626 can generally govern and manage the user input and system output. There is no restriction on operating on any particular hardware arrangement and therefore the basic features here may easily be substituted for improved hardware or firmware arrangements as they are developed.

Storage device 608 is a non-volatile memory and can be a hard disk or other types of computer readable media which can store data that are accessible by a computer, such as magnetic cassettes, flash memory cards, solid state memory devices, digital versatile disks, cartridges, RAMs 616, ROM 618, and hybrids thereof.

The storage device 608 can include services 610, 612, 614 for controlling the processor 604. Other hardware or software modules are contemplated. The storage device 608 can be connected to the system connection 606. In one aspect, a hardware module that performs a particular function can include the software component stored in a computer-readable medium in connection with the necessary hardware components, such as the processor 604, connection 606, output device 624, and so forth, to carry out the function.

The disclosed methods can be performed using a computing system. An example computing system can include a processor (e.g., a central processing unit), memory, non-volatile memory, and an interface device. The memory may store data and/or and one or more code sets, software, scripts, etc. The components of the computer system can be coupled together via a bus or through some other known or convenient device. The processor may be configured to carry out all or part of methods described herein for example by executing code for example stored in memory. One or more of a user device or computer, a provider server or system, or a suspended database update system may include the components of the computing system or variations on such a system.

This disclosure contemplates the computer system taking any suitable physical form, including, but not limited to a Point-of-Sale system (“POS”). As example and not by way of limitation, the computer system may be an embedded computer system, a system-on-chip (SOC), a single-board computer system (SBC) (such as, for example, a computer-on-module (COM) or system-on-module (SOM)), a desktop computer system, a laptop or notebook computer system, an interactive kiosk, a mainframe, a mesh of computer systems, a mobile telephone, a personal digital assistant (PDA), a server, or a combination of two or more of these. Where appropriate, the computer system may include one or more computer systems; be unitary or distributed; span multiple locations; span multiple machines; and/or reside in a cloud, which may include one or more cloud components in one or more networks. Where appropriate, one or more computer systems may perform without substantial spatial or temporal limitation one or more steps of one or more methods described or illustrated herein. As an example and not by way of limitation, one or more computer systems may perform in real time or in batch mode one or more steps of one or more methods described or illustrated herein. One or more computer systems may perform at different times or at different locations one or more steps of one or more methods described or illustrated herein, where appropriate.

The processor may be, for example, be a conventional microprocessor such as an Intel Pentium microprocessor or Motorola power PC microprocessor. One of skill in the relevant art will recognize that the terms “machine-readable (storage) medium” or “computer-readable (storage) medium” include any type of device that is accessible by the processor.

The memory can be coupled to the processor by, for example, a bus. The memory can include, by way of example but not limitation, random access memory (RAM), such as dynamic RAM (DRAM) and static RAM (SRAM). The memory can be local, remote, or distributed.

The bus can also couple the processor to the non-volatile memory and drive unit. The non-volatile memory is often a magnetic floppy or hard disk, a magnetic-optical disk, an optical disk, a read-only memory (ROM), such as a CD-ROM, EPROM, or EEPROM, a magnetic or optical card, or another form of storage for large amounts of data. Some of this data is often written, by a direct memory access process, into memory during execution of software in the computer. The non-volatile storage can be local, remote, or distributed. The non-volatile memory is optional because systems can be created with all applicable data available in memory. A typical computer system will usually include at least a processor, memory, and a device (e.g., a bus) coupling the memory to the processor.

Software can be stored in the non-volatile memory and/or the drive unit. Indeed, for large programs, it may not even be possible to store the entire program in the memory. Nevertheless, it should be understood that for software to run, if necessary, it is moved to a computer readable location appropriate for processing, and for illustrative purposes, that location is referred to as the memory herein. Even when software is moved to the memory for execution, the processor can make use of hardware registers to store values associated with the software, and local cache that, ideally, serves to speed up execution. As used herein, a software program is assumed to be stored at any known or convenient location (from non-volatile storage to hardware registers), when the software program is referred to as “implemented in a computer-readable medium.” A processor is considered to be “configured to execute a program” when at least one value associated with the program is stored in a register readable by the processor.

The bus can also couple the processor to the network interface device. The interface can include one or more of a modem or network interface. It will be appreciated that a modem or network interface can be considered to be part of the computer system. The interface can include an analog modem, Integrated Services Digital network (ISDNO modem, cable modem, token ring interface, satellite transmission interface (e.g., “direct PC”), or other interfaces for coupling a computer system to other computer systems. The interface can include one or more input and/or output (I/O) devices. The I/O devices can include, by way of example but not limitation, a keyboard, a mouse or other pointing device, disk drives, printers, a scanner, and other input and/or output devices, including a display device. The display device can include, by way of example but not limitation, a cathode ray tube (CRT), liquid crystal display (LCD), or some other applicable known or convenient display device.

In operation, the computer system can be controlled by operating system software that includes a file management system, such as a disk operating system. One example of operating system software with associated file management system software is the family of operating systems known as Windows® from Microsoft Corporation of Redmond, WA, and their associated file management systems. Another example of operating system software with its associated file management system software is the Linux™ operating system and its associated file management system. The file management system can be stored in the non-volatile memory and/or drive unit and can cause the processor to execute the various acts required by the operating system to input and output data and to store data in the memory, including storing files on the non-volatile memory and/or drive unit.

Some portions of the detailed description may be presented in terms of algorithms and symbolic representations of operations on data bits within a computer memory. These algorithmic descriptions and representations are the means used by those skilled in the data processing arts to most effectively convey the substance of their work to others skilled in the art. An algorithm is here, and generally, conceived to be a self-consistent sequence of operations leading to a desired result. The operations are those requiring physical manipulations of physical quantities. Usually, though not necessarily, these quantities take the form of electrical or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind, however, that all of these and similar terms are to be associated with the appropriate physical quantities and are merely convenient labels applied to these quantities. Unless specifically stated otherwise as apparent from the following discussion, it is appreciated that throughout the description, discussions utilizing terms such as “processing” or “computing” or “calculating” or “determining” or “displaying” or “generating” or the like, refer to the action and processes of a computer system, or similar electronic computing device, that manipulates and transforms data represented as physical (electronic) quantities within registers and memories of the computer system into other data similarly represented as physical quantities within the computer system memories or registers or other such information storage, transmission or display devices.

The algorithms and displays presented herein are not inherently related to any particular computer or other apparatus. Various general purpose systems may be used with programs in accordance with the teachings herein, or it may prove convenient to construct more specialized apparatus to perform the methods of some examples. The required structure for a variety of these systems will appear from the description below. In addition, the techniques are not described with reference to any particular programming language, and various examples may thus be implemented using a variety of programming languages.

In various implementations, the system operates as a standalone device or may be connected (e.g., networked) to other systems. In a networked deployment, the system may operate in the capacity of a server or a client system in a client-server network environment, or as a peer system in a peer-to-peer (or distributed) network environment.

The system may be a server computer, a client computer, a personal computer (PC), a tablet PC, a laptop computer, a set-top box (STB), a personal digital assistant (PDA), a cellular telephone, an iPhone, a Blackberry, a processor, a telephone, a web appliance, a network router, switch or bridge, or any system capable of executing a set of instructions (sequential or otherwise) that specify actions to be taken by that system.

While the machine-readable medium or machine-readable storage medium is shown, by way of example, to be a single medium, the term “machine-readable medium” and “machine-readable storage medium” should be taken to include a single medium or multiple media (e.g., a centralized or distributed database, and/or associated caches and servers) that store the one or more sets of instructions. The term “machine-readable medium” and “machine-readable storage medium” shall also be taken to include any medium that is capable of storing, encoding, or carrying a set of instructions for execution by the system and that cause the system to perform any one or more of the methodologies or modules of disclosed herein.

In general, the routines executed to implement the implementations of the disclosure, may be implemented as part of an operating system or a specific application, component, program, object, module or sequence of instructions referred to as “computer programs.” The computer programs typically comprise one or more instructions set at various times in various memory and storage devices in a computer, and that, when read and executed by one or more processing units or processors in a computer, cause the computer to perform operations to execute elements involving the various aspects of the disclosure.

Moreover, while examples have been described in the context of fully functioning computers and computer systems, those skilled in the art will appreciate that the various examples are capable of being distributed as a program object in a variety of forms, and that the disclosure applies equally regardless of the particular type of machine or computer-readable media used to actually effect the distribution.

Further examples of machine-readable storage media, machine-readable media, or computer-readable (storage) media include but are not limited to recordable type media such as volatile and non-volatile memory devices, floppy and other removable disks, hard disk drives, optical disks (e.g., Compact Disk Read-Only Memory (CD ROMS), Digital Versatile Disks, (DVDs), etc.), among others, and transmission type media such as digital and analog communication links.

In some circumstances, operation of a memory device, such as a change in state from a binary one to a binary zero or vice-versa, for example, may comprise a transformation, such as a physical transformation. With particular types of memory devices, such a physical transformation may comprise a physical transformation of an article to a different state or thing. For example, but without limitation, for some types of memory devices, a change in state may involve an accumulation and storage of charge or a release of stored charge. Likewise, in other memory devices, a change of state may comprise a physical change or transformation in magnetic orientation or a physical change or transformation in molecular structure, such as from crystalline to amorphous or vice versa. The foregoing is not intended to be an exhaustive list of all examples in which a change in state for a binary one to a binary zero or vice-versa in a memory device may comprise a transformation, such as a physical transformation. Rather, the foregoing is intended as illustrative examples.

A storage medium typically may be non-transitory or comprise a non-transitory device. In this context, a non-transitory storage medium may include a device that is tangible, meaning that the device has a concrete physical form, although the device may change its physical state. Thus, for example, non-transitory refers to a device remaining tangible despite this change in state.

The above description and drawings are illustrative and are not to be construed as limiting the subject matter to the precise forms disclosed. Persons skilled in the relevant art can appreciate that many modifications and variations are possible in light of the above disclosure. Numerous specific details are described to provide a thorough understanding of the disclosure. However, in certain instances, well-known or conventional details are not described in order to avoid obscuring the description.

As used herein, the terms “connected,” “coupled,” or any variant thereof when applying to modules of a system, means any connection or coupling, either direct or indirect, between two or more elements; the coupling of connection between the elements can be physical, logical, or any combination thereof. Additionally, the words “herein,” “above,” “below,” and words of similar import, when used in this application, shall refer to this application as a whole and not to any particular portions of this application. Where the context permits, words in the above Detailed Description using the singular or plural number may also include the plural or singular number respectively. The word “or,” in reference to a list of two or more items, covers all of the following interpretations of the word: any of the items in the list, all of the items in the list, or any combination of the items in the list.

Those of skill in the art will appreciate that the disclosed subject matter may be embodied in other forms and manners not shown below. It is understood that the use of relational terms, if any, such as first, second, top and bottom, and the like are used solely for distinguishing one entity or action from another, without necessarily requiring or implying any such actual relationship or order between such entities or actions.

While processes or blocks are presented in a given order, alternative implementations may perform routines having steps, or employ systems having blocks, in a different order, and some processes or blocks may be deleted, moved, added, subdivided, substituted, combined, and/or modified to provide alternative or sub combinations. Each of these processes or blocks may be implemented in a variety of different ways. Also, while processes or blocks are at times shown as being performed in series, these processes or blocks may instead be performed in parallel, or may be performed at different times. Further any specific numbers noted herein are only examples: alternative implementations may employ differing values or ranges.

The teachings of the disclosure provided herein can be applied to other systems, not necessarily the system described above. The elements and acts of the various examples described above can be combined to provide further examples.

Any patents and applications and other references noted above, including any that may be listed in accompanying filing papers, are incorporated herein by reference. Aspects of the disclosure can be modified, if necessary, to employ the systems, functions, and concepts of the various references described above to provide yet further examples of the disclosure.

These and other changes can be made to the disclosure in light of the above Detailed Description. While the above description describes certain examples, and describes the best mode contemplated, no matter how detailed the above appears in text, the teachings can be practiced in many ways. Details of the system may vary considerably in its implementation details, while still being encompassed by the subject matter disclosed herein. As noted above, particular terminology used when describing certain features or aspects of the disclosure should not be taken to imply that the terminology is being redefined herein to be restricted to any specific characteristics, features, or aspects of the disclosure with which that terminology is associated. In general, the terms used in the following claims should not be construed to limit the disclosure to the specific implementations disclosed in the specification, unless the above Detailed Description section explicitly defines such terms. Accordingly, the actual scope of the disclosure encompasses not only the disclosed implementations, but also all equivalent ways of practicing or implementing the disclosure under the claims.

While certain aspects of the disclosure are presented below in certain claim forms, the inventors contemplate the various aspects of the disclosure in any number of claim forms. Any claims intended to be treated under 35 U.S.C. § 112(f) will begin with the words “means for”. Accordingly, the applicant reserves the right to add additional claims after filing the application to pursue such additional claim forms for other aspects of the disclosure.

The terms used in this specification generally have their ordinary meanings in the art, within the context of the disclosure, and in the specific context where each term is used.

Certain terms that are used to describe the disclosure are discussed above, or elsewhere in the specification, to provide additional guidance to the practitioner regarding the description of the disclosure. For convenience, certain terms may be highlighted, for example using capitalization, italics, and/or quotation marks. The use of highlighting has no influence on the scope and meaning of a term; the scope and meaning of a term is the same, in the same context, whether or not it is highlighted. It will be appreciated that same element can be described in more than one way.

Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of other synonyms. The use of examples anywhere in this specification including examples of any terms discussed herein is illustrative only, and is not intended to further limit the scope and meaning of the disclosure or of any exemplified term. Likewise, the disclosure is not limited to various examples given in this specification.

Client devices, network devices, and other devices can be computing systems that include one or more integrated circuits, input devices, output devices, data storage devices, and/or network interfaces, among other things. The integrated circuits can include, for example, one or more processors, volatile memory, and/or non-volatile memory, among other things. The input devices can include, for example, a keyboard, a mouse, a key pad, a touch interface, a microphone, a camera, and/or other types of input devices. The output devices can include, for example, a display screen, a speaker, a haptic feedback system, a printer, and/or other types of output devices. A data storage device, such as a hard drive or flash memory, can enable the computing device to temporarily or permanently store data. A network interface, such as a wireless or wired interface, can enable the computing device to communicate with a network. Examples of computing devices include desktop computers, laptop computers, server computers, hand-held computers, tablets, smart phones, personal digital assistants, digital home assistants, as well as machines and apparatuses in which a computing device has been incorporated.

The term “computer-readable medium” includes, but is not limited to, portable or non-portable storage devices, optical storage devices, and various other mediums capable of storing, containing, or carrying instruction(s) and/or data. A computer-readable medium may include a non-transitory medium in which data can be stored and that does not include carrier waves and/or transitory electronic signals propagating wirelessly or over wired connections. Examples of a non-transitory medium may include, but are not limited to, a magnetic disk or tape, optical storage media such as compact disk (CD) or digital versatile disk (DVD), flash memory, memory or memory devices. A computer-readable medium may have stored thereon code and/or machine-executable instructions that may represent a procedure, a function, a subprogram, a program, a routine, a subroutine, a module, a software package, a class, or any combination of instructions, data structures, or program statements. A code segment may be coupled to another code segment or a hardware circuit by passing and/or receiving information, data, arguments, parameters, or memory contents. Information, arguments, parameters, data, etc. may be passed, forwarded, or transmitted via any suitable means including memory sharing, message passing, token passing, network transmission, or the like.

The various examples discussed above may further be implemented by hardware, software, firmware, middleware, microcode, hardware description languages, or any combination thereof. When implemented in software, firmware, middleware or microcode, the program code or code segments to perform the necessary tasks (e.g., a computer-program product) may be stored in a computer-readable or machine-readable storage medium (e.g., a medium for storing program code or code segments). A processor(s), implemented in an integrated circuit, may perform the necessary tasks.

Where components are described as being “configured to” perform certain operations, such configuration can be accomplished, for example, by designing electronic circuits or other hardware to perform the operation, by programming programmable electronic circuits (e.g., microprocessors, or other suitable electronic circuits) to perform the operation, or any combination thereof.

The various illustrative logical blocks, modules, circuits, and algorithm steps described in connection with the implementations disclosed herein may be implemented as electronic hardware, computer software, firmware, or combinations thereof. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present disclosure.

The techniques described herein may also be implemented in electronic hardware, computer software, firmware, or any combination thereof. Such techniques may be implemented in any of a variety of devices such as general purposes computers, wireless communication device handsets, or integrated circuit devices having multiple uses including application in wireless communication device handsets and other devices. Any features described as modules or components may be implemented together in an integrated logic device or separately as discrete but interoperable logic devices. If implemented in software, the techniques may be realized at least in part by a computer-readable data storage medium comprising program code including instructions that, when executed, performs one or more of the methods described above. The computer-readable data storage medium may form part of a computer program product, which may include packaging materials. The computer-readable medium may comprise memory or data storage media, such as random access memory (RAM) such as synchronous dynamic random access memory (SDRAM), read-only memory (ROM), non-volatile random access memory (NVRAM), electrically erasable programmable read-only memory (EEPROM), FLASH memory, magnetic or optical data storage media, and the like. The techniques additionally, or alternatively, may be realized at least in part by a computer-readable communication medium that carries or communicates program code in the form of instructions or data structures and that can be accessed, read, and/or executed by a computer, such as propagated signals or waves.

The program code may be executed by a processor, which may include one or more processors, such as one or more digital signal processors (DSPs), general purpose microprocessors, an application specific integrated circuits (ASICs), field programmable logic arrays (FPGAs), or other equivalent integrated or discrete logic circuitry. Such a processor may be configured to perform any of the techniques described in this disclosure. A general purpose processor may be a microprocessor; but in the alternative, the processor may be any conventional processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration. Accordingly, the term “processor,” as used herein may refer to any of the foregoing structure, any combination of the foregoing structure, or any other structure or apparatus suitable for implementation of the techniques described herein. In addition, in some aspects, the functionality described herein may be provided within dedicated software modules or hardware modules configured for implementing a suspended database update system.

One aspect of the instant disclosure encompasses a computing system for detecting compounds with weak signals in mass spectrometry (MS) data. The computing system comprises: (a) a communication interface that receives EICs of portions of each of a plurality of MS chromatograms, wherein the portions of the plurality of chromatograms bear the same m/z value; and (b) a processor that executes instructions stored in memory, wherein the processor executes the instructions to: (i) identify a first set of compounds from a merged mass spectrum of m/z signal intensities, wherein the first set of compounds is identified based on a concentration of signals corresponding to mass measurements associated with the first set of compounds and isotopologues of the first set of compounds; (ii) calculate a signal bandwidth for identification of a second set of compounds, wherein the signal bandwidth is calculated based on a limit of detection associated with the mass spectrometer and a lowest signal intensity corresponding to an isotopologue associated with the first set of compounds; and (iii) identify a set of signal intensity adjacencies within the signal bandwidth, wherein the set of signal intensity adjacencies are indicative of a second set of compounds; (iv) optionally filter signals associated with the first set of compounds from the merged mass spectrum prior to identifying the set of signal intensity adjacencies within the signal bandwidth; and (v) optionally iteratively repeat steps (i) to (iv). In some embodiments, the compound is metabolites, isomers of metabolites, or both. In some embodiments, the chromatography-MS is liquid chromatography-MS (LC-MS). The ceiling for this ideal signal bandwidth can be dynamically calculated by identifying the signal intensity corresponding to the least abundant metabolite that is part of a valid isopair. In some embodiments, the processor executes the instructions to receive the plurality of chromatography-MS mass spectra.

In some embodiments, the processor further executes the instructions to: divide the merged mass spectrum within the signal bandwidth into bins. The bins can be sized to detect the set of signal intensity adjacencies within the signal bandwidth. In some embodiments, the bins correspond to an atomic mass range equivalent to 1/10,000th Daltons.

The processor can further execute the instructions to: identify one or more inorganic salts from the merged mass spectrum. The one or more inorganic salts can be identified as a result of signal intensities corresponding to the one or more inorganic salts exceeding a required lowest signal intensity corresponding to an expected isotopologue, while there is no expected isotopologue detected.

In some embodiments, the set of signal intensity adjacencies are identified according to statistical differences between known metabolite signal intensities and electrostatic noise signal intensities.

In some embodiments, the second set of compounds is identified without requiring identification of corresponding isotopologues from the merged mass spectrum.

Another aspect of the instant disclosure encompasses a non-transitory, computer-readable storage medium, having embodied thereon a program executable by a processor to perform a method of detecting compounds with weak signals in mass spectrometry (MS) data. The method comprises (a) combining or having combined a plurality of chromatography-mass spectrometry (chromatography-MS) mass spectra comprising mass-to-charge (m/z) signal intensities associated with mass measurements of compounds into a merged mass spectrum of m/z signal intensities; (b) identifying a first set of compounds from the merged mass spectrum, wherein the first set of compounds is identified based on a concentration of signals corresponding to mass measurements associated with the first set of compounds and isotopologues of the first set of compounds; (c) calculating a signal bandwidth for identification of a second set of compounds, wherein the signal bandwidth is calculated based on a limit of detection associated with the mass spectrometer and a lowest signal intensity corresponding to an isotopologue associated with the first set of compounds; and (d) identifying a set of signal intensity adjacencies within the signal bandwidth, wherein the set of signal intensity adjacencies are indicative of a second set of compounds. The method can optionally comprise filtering signals associated with the first set of compounds from the merged mass spectrum prior to identifying the set of signal intensity adjacencies within the signal bandwidth. The method can also optionally comprise iteratively repeating steps (b) to (d).

Importantly, the second set of compounds can be identified without requiring identification of corresponding isotopologues from the merged mass spectrum.

In some embodiments, the method further comprises receiving or having received the plurality of chromatography-MS mass spectra. In some embodiments, the compound is metabolites, isomers of metabolites, or both. Additionally, the chromatography-MS can be liquid chromatography-MS (LC-MS).

In some embodiments, a method of the instant disclosure further comprises dividing the merged mass spectrum within the signal bandwidth into bins, wherein the bins are sized to detect the set of signal intensity adjacencies within the signal bandwidth. In some embodiments, the set of signal intensity adjacencies are identified according to statistical differences between known metabolite signal intensities and electrostatic noise signal intensities. In some embodiments, the bins correspond to an atomic mass range equivalent to 1/10,000th Daltons.

The method can also further comprise identifying one or more inorganic salts from the merged mass spectrum, wherein the one or more inorganic salts are identified as a result of signal intensities corresponding to the one or more inorganic salts exceeding a required lowest signal intensity corresponding to an expected isotopologue, while there is no expected isotopologue detected.

In some embodiments, the set of signal intensity adjacencies are identified according to statistical differences between known metabolite signal intensities and electrostatic noise signal intensities. Further, the second set of compounds can be identified without requiring identification of corresponding isotopologues from the merged mass spectrum.

### Definitions

Unless defined otherwise, all technical and scientific terms used herein have the meaning commonly understood by a person skilled in the art to which this invention belongs. As used herein, the following terms have the meanings ascribed to them unless specified otherwise.

Some portions of this description describe examples in terms of algorithms and symbolic representations of operations on information. These algorithmic descriptions and representations are commonly used by those skilled in the data processing arts to convey the substance of their work effectively to others skilled in the art. These operations, while described functionally, computationally, or logically, are understood to be implemented by computer programs or equivalent electrical circuits, microcode, or the like. Furthermore, it has also proven convenient at times, to refer to these arrangements of operations as modules, without loss of generality. The described operations and their associated modules may be embodied in software, firmware, hardware, or any combinations thereof.

Without intent to further limit the scope of the disclosure, examples of instruments, apparatus, methods and their related results according to the examples of the present disclosure are given below. Note that titles or subtitles may be used in the examples for convenience of a reader, which in no way should limit the scope of the disclosure. Unless otherwise defined, all technical and scientific terms used herein have the same meaning as commonly understood by one of ordinary skill in the art to which this disclosure pertains. In the case of conflict, the present document, including definitions will control.

The language used in the specification has been principally selected for readability and instructional purposes, and it may not have been selected to delineate or circumscribe the subject matter. It is therefore intended that the scope of this disclosure be limited not by this detailed description, but rather by any claims that issue on an application based hereon. Accordingly, the disclosure of the examples is intended to be illustrative, but not limiting, of the scope of the subject matter, which is set forth in the following claims.

Specific details were given in the preceding description to provide a thorough understanding of various implementations of systems and components for a contextual connection system. It will be understood by one of ordinary skill in the art, however, that the implementations described above may be practiced without these specific details. For example, circuits, systems, networks, processes, and other components may be shown as components in block diagram form in order not to obscure the embodiments in unnecessary detail. In other instances, well-known circuits, processes, algorithms, structures, and techniques may be shown without unnecessary detail in order to avoid obscuring the embodiments.

It is also noted that individual implementations may be described as a process which is depicted as a flowchart, a flow diagram, a data flow diagram, a structure diagram, or a block diagram. Although a flowchart may describe the operations as a sequential process, many of the operations can be performed in parallel or concurrently. In addition, the order of the operations may be re-arranged. A process is terminated when its operations are completed but could have additional steps not included in a figure. A process may correspond to a method, a function, a procedure, a subroutine, a subprogram, etc. When a process corresponds to a function, its termination can correspond to a return of the function to the calling function or the main function.

The foregoing detailed description of the technology has been presented for purposes of illustration and description. It is not intended to be exhaustive or to limit the technology to the precise form disclosed. Many modifications and variations are possible in light of the above teaching. The described embodiments were chosen in order to best explain the principles of the technology, its practical application, and to enable others skilled in the art to utilize the technology in various embodiments and with various modifications as are suited to the particular use contemplated. It is intended that the scope of the technology be defined by the claim.

When introducing elements of the present disclosure or the preferred aspects(s) thereof, the articles “a”, “an”, “the” and “said” are intended to mean that there are one or more of the elements. The terms “comprising”, “including” and “having” are intended to be inclusive and mean that there may be additional elements other than the listed elements.

As various changes could be made in the above-described cells and methods without departing from the scope of the invention, it is intended that all matter contained in the above description and in the examples given below, shall be interpreted as illustrative and not in a limiting sense.

What is claimed is:

1. A method for detecting compounds with weak signals in mass spectrometry (MS) data, the method comprising:
a. combining or having combined a plurality of chromatography-mass spectrometry (chromatography-MS) mass spectra comprising mass-to-charge (m/z) signal intensities associated with mass measurements of compounds into a merged mass spectrum of m/z signal intensities;
b. identifying a first set of compounds from the merged mass spectrum, wherein the first set of compounds is identified based on a concentration of signals corresponding to mass measurements associated with the first set of compounds and isotopologues of the first set of compounds;
c. calculating a signal bandwidth for identification of a second set of compounds, wherein the signal bandwidth is calculated based on a limit of detection associated with the mass spectrometer and a lowest signal intensity corresponding to an isotopologue associated with the first set of compounds; and
d. identifying a set of signal intensity adjacencies within the signal bandwidth, wherein the set of signal intensity adjacencies are indicative of a second set of compounds;
e. optionally filtering signals associated with the first set of compounds from the merged mass spectrum prior to identifying the set of signal intensity adjacencies within the signal bandwidth; and
f. optionally iteratively repeating steps (b) to (d).

2. The method of claim 1, wherein the compound is metabolites, isomers of metabolites, or both.

3. The method of claim 1, wherein the chromatography-MS is liquid chromatography-MS (LC-MS).

4. The method of claim 1, wherein the ceiling for this ideal signal bandwidth is dynamically calculated by identifying the signal intensity corresponding to the least abundant metabolite that is part of a valid isopair.

5. The method of claim 1, further comprising: dividing the merged mass spectrum within the signal bandwidth into bins, wherein the bins are sized to detect the set of signal intensity adjacencies within the signal bandwidth.

6. The method of claim 5, wherein the bins correspond to an atomic mass range equivalent to 1/10,000th Daltons.

7. The method of claim 1, further comprising: identifying one or more inorganic salts from the merged mass spectrum, wherein the one or more inorganic salts are identified as a result of signal intensities corresponding to the one or more inorganic salts exceeding a required lowest signal intensity corresponding to an expected isotopologue, while there is no expected isotopologue detected.

8. The method of claim 1, wherein the set of signal intensity adjacencies are identified according to statistical differences between known metabolite signal intensities and electrostatic noise signal intensities.

9. The method of claim 1, wherein the second set of compounds is identified without requiring identification of corresponding isotopologues from the merged mass spectrum.

10. The method of claim 1, further comprising receiving or having received the plurality of chromatography-MS mass spectra.

11. A computing system for detecting compounds with weak signals in mass spectrometry (MS) data. The computing system comprising:
a. a communication interface that receives EICs of portions of each of a plurality of MS chromatograms, wherein the portions of the plurality of chromatograms bear the same m/z value; and
b. a processor that executes instructions stored in memory, wherein the processor executes the instructions to:
i. identify a first set of compounds from a merged mass spectrum of m/z signal intensities, wherein the first set of compounds is identified based on a concentration of signals corresponding to mass measurements associated with the first set of compounds and isotopologues of the first set of compounds;
ii. calculate a signal bandwidth for identification of a second set of compounds, wherein the signal bandwidth is calculated based on a limit of detection associated with the mass spectrometer and a lowest signal intensity corresponding to an isotopologue associated with the first set of compounds; and
iii. identify a set of signal intensity adjacencies within the signal bandwidth, wherein the set of signal intensity adjacencies are indicative of a second set of compounds;
iv. optionally filter signals associated with the first set of compounds from the merged mass spectrum prior to identifying the set of signal intensity adjacencies within the signal bandwidth; and
v. optionally iteratively repeat steps (i) to (iv).

12-14. (canceled)

15. The computing system of claim 11, wherein the processor further executes the instructions to: divide the merged mass spectrum within the signal bandwidth into bins, wherein the bins are sized to detect the set of signal intensity adjacencies within the signal bandwidth.

16. (canceled)

17. The computing system of claim 11, wherein the processor further executes the instructions to: identify one or more inorganic salts from the merged mass spectrum, wherein the one or more inorganic salts are identified as a result of signal intensities corresponding to the one or more inorganic salts exceeding a required lowest signal intensity corresponding to an expected isotopologue, while there is no expected isotopologue detected.

18-20. (canceled)

21. A non-transitory, computer-readable storage medium, having embodied thereon a program executable by a processor to perform a method of detecting compounds with weak signals in mass spectrometry (MS) data, the method comprising, the method comprising:
a. combining or having combined a plurality of chromatography-mass spectrometry (chromatography-MS) mass spectra comprising mass-to-charge (m/z) signal intensities associated with mass measurements of compounds into a merged mass spectrum of m/z signal intensities;
b. identifying a first set of compounds from the merged mass spectrum, wherein the first set of compounds is identified based on a concentration of signals corresponding to mass measurements associated with the first set of compounds and isotopologues of the first set of compounds; calculating a signal bandwidth for identification of a second set of compounds, wherein the signal bandwidth is calculated based on a limit of detection associated with the mass spectrometer and a lowest signal intensity corresponding to an isotopologue associated with the first set of compounds; and
c. identifying a set of signal intensity adjacencies within the signal bandwidth, wherein the set of signal intensity adjacencies are indicative of a second set of compounds;
d. optionally filtering signals associated with the first set of compounds from the merged mass spectrum prior to identifying the set of signal intensity adjacencies within the signal bandwidth; and
e. optionally iteratively repeating steps (b) to (d).

22-24. (canceled)

25. The non-transitory, computer-readable storage medium of claim 21, further comprising: dividing the merged mass spectrum within the signal bandwidth into bins, wherein the bins are sized to detect the set of signal intensity adjacencies within the signal bandwidth.

26. (canceled)

27. The non-transitory, computer-readable storage medium of claim 21, further comprising: identifying one or more inorganic salts from the merged mass spectrum, wherein the one or more inorganic salts are identified as a result of signal intensities corresponding to the one or more inorganic salts exceeding a required lowest signal intensity corresponding to an expected isotopologue, while there is no expected isotopologue detected.

28-30. (canceled)
